(19) United States
(12) Patent Application Publication
(10) Pub. No.: US 2011/0286924 A1
(43) Pub. Date: Nov. 24, 2011

(54) NOVEL HUMAN ENDOGENOUS RETROVIRAL ERV3 VARIANT AND USES THEREOF IN THE DIAGNOSING OVARIAN CANCER

(75) Inventors: Gilles Bernard Tremblay, La Prairie (CA); Mario Filion, Longueuil (CA); Anna Moraitis, Laval (CA)

(73) Assignee: ALETHIA BIOTHERAPEUTICS INC, MONTREAL (CA)

(21) Appl. No.: 12/998,826

(22) PCT Filed: Dec. 23, 2008
(86) PCT No.: PCT/CA2008/002289

§ 371 Date: Aug. 10, 2011

Related U.S. Application Data
(60) Provisional application No. 61/193,597, filed on Dec. 9, 2008.

(51) Int. Cl. A61K 49/00; A61P 35/00; A61K 31/7088; C07K 16/18; A61K 39/395; A61K 38/00

(52) U.S. Cl. 424/9.1; 424/139.1; 424/174.1; 514/19.3; 514/44 R; 530/389.7; 530/391.3; 530/391.7

(57) ABSTRACT

The present invention relates to polynucleotide and polypeptide sequences of a novel human endogenous retroviral ERV3 variant which is differentially expressed in ovarian cancer cells when compared to normal cells. The present invention more particularly relates to the use of these polynucleotide and polypeptide in the diagnosis, prognosis or treatment of cancer and in the detection of cancer cells.

### FIELD OF THE INVENTION

The present invention relates to polynucleotide and polypeptide sequences which are differentially expressed in cancer cells compared to normal cells. The present invention more particularly relates to the use of these sequences and reagents specifically binding to these sequences in the diagnosis, prognosis or treatment of cancer and in the detection of cancer cells.

### BACKGROUND OF THE INVENTION

Among gynecologic malignancies, ovarian cancer accounts for the highest tumor-related mortality in women in the United States (Jemal et al., 2005). It is the fourth leading cause of cancer-related death in women in the U.S (Menon et al., 2005). The American Cancer Society estimated a total of 22,220 new cases in 2005 and attributed 16,210 deaths to the disease (Bonome et al., 2005). For the past 30 years, the statistics have remained largely the same—the majority of women who develop ovarian cancer will die of this disease (Chambers and Vanderhyden, 2006). The disease carries a 1:70 lifetime risk and a mortality rate of >60% (Chambers and Vanderhyden, 2006). The high mortality rate is due to the difficulties with the early detection of ovarian cancer when the malignancy has already spread beyond the ovary. Indeed, >80% of patients are diagnosed with advanced staged disease (stage III or IV) (Bonome et al., 2005). These patients have a poor prognosis that is reflected in <45% 5-year survival rate, although 80% to 90% will initially respond to chemotherapy (Berek et al., 2000). This increased success compared to 20% 5-year survival rate years earlier is, at least in part, due to the ability to optimally debulk tumor tissue when it is confined to the ovaries, which is a significant prognostic factor for ovarian cancer (Bristow R. E., 2000 and Brown et al., 2004). In patients who are diagnosed with early disease (stage I), the 5-yr survival ranges from >90 (Chambers and Vanderhyden, 2006).

Ovarian cancer comprises a heterogeneous group of tumors that are derived from the surface epithelium of the ovary or from surface inclusions. They are classified into serous, mucinous, endometrioid, clear cell, and Brenner (transitional) types corresponding to the different types of epithelia in the organs of the female reproductive tract (Shih and Kurman, 2005). Of these, serous tumors account for ˜60% of the ovarian cancer cases diagnosed. Each histologic subcategory is further divided into three groups: benign, intermediate (borderline tumor or low malignancy potential (LMP)), and malignant, reflecting their clinical behavior (Seidman et al., 2002). LMP represents 10% to 15% of tumors diagnosed as serous and is a conundrum as they display atypical nuclear structure and metastatic behavior, yet they are considerably less aggressive than high-grade serous tumors. The 5-year survival for patients with LMP tumors is 95% in contrast to a <45% survival for advanced high-grade disease over the same period (Berek et al., 2000).

Despite improved knowledge of the etiology of the disease, aggressive cytoreductive surgery, and modern combination chemotherapy, there has been only little change in mortality. Poor outcomes have been attributed to (1) lack of adequate screening tests for early disease detection, in combination with only subtle presentation of symptoms at this stage—diagnosis is frequently being made only after progression to later stages, at which point the peritoneal dissemination of the cancer limits effective treatment and (2) the frequent development of resistance to standard chemotherapeutic strategies limiting improvement in the 5-year survival rate of patients. The initial chemotherapy regimen for ovarian cancer includes the combination of carboplatin (Paraplatin) and paclitaxel (taxol). Years of clinical trials have proved this combination to be most effective after effective surgery—reduces tumor volume in about 80% of the women with newly diagnosed ovarian cancer and 40% to 50% will have complete regression—but studies continue to look for ways to improve it. Recent abdominal infusion of chemotherapeutics to target hard-to-reach cells in combination with intravenous delivery has increased the effectiveness. However, severe side effects often lead to an incomplete course of treatment. Some other chemotherapeutic agents include doxorubicin, cisplatin, cyclophosphamide, bleomycin, etoposide, vinblastine, topotecan hydrochloride, ifosfamide, 5-fluorouracil and melphalan. The excellent survival rates for women with early stage disease receiving chemotherapy provide a strong rationale for research efforts to develop strategies to improve the detection of ovarian cancer. Furthermore, the discovery of new ovarian cancer-related biomarkers will lead to the development of more effective therapeutic strategies with minimal side effects for the future treatment of ovarian cancer.

Presently, the diagnosis of ovarian cancer is accomplished, in part, through routine analysis of the medical history of patients and by performing physical, ultrasound and x-ray examinations, and hematological screening. Two alternative strategies have been reported for early hematological detection of serum biomarkers. One approach is the analysis of serum samples by mass spectrometry to find proteins or protein fragments of unknown identity that detect the presence or absence of cancer (Mor et al., 2005 and Kozak et al., 2003). However, this strategy is expensive and not broadly available. Alternatively, the presence or absence of known proteins/peptides in the serum is being detected using antibody microarrays, ELISA, or other similar approaches. Serum testing for a protein biomarker called CA-125 (cancer antigen-125) has long been widely performed as a marker for ovarian cancer. However, although ovarian cancer cells may produce an excess of these protein molecules, there are some other cancers, including cancer of the fallopian tube or endometrial cancer (cancer of the lining of the uterus), 60% of people with pancreatic cancer, and 20%-25% of people with other malignancies with elevated levels of CA-125. The CA-125 test only returns a true positive result for about 50% of Stage I ovarian cancer patients and has a 80% chance of returning true positive results from stage 11, III, and IV ovarian cancer patients. The other 20% of ovarian cancer patients do not show any increase in CA-125 concentrations. In addition, an elevated CA-125 test may indicate other benign activity not associated with cancer, such as menstruation, pregnancy, or endometriosis. Consequently, this test has very limited clinical application for the detection of early stage disease when it is still treatable, exhibiting a positive predictive value (PPV) of <10%. And, even with the addition of ultrasound screening to CA-125, the PPV only improves to around 20% (Kozak et al., 2003). Thus, this test is not an effective screening test.

Other studies have yielded a number of biomarker combinations with increased specificity and sensitivity for ovarian cancer relative to CA-125 alone (McIntosh et al., 2004, Woolas et al., 1993, Schorge et., 2004). Serum biomarkers that are often elevated in women with epithelial ovarian cancer, but not exclusively, include carcinoembryonic antigen, ovarian cystadenocarcinoma antigen, lipidassociated sialic acid, NB/70, TAG72.3, CA-15.3, and CA-125. Unfortunately, although this approach has increased the sensitivity and specificity of early detection, published biomarker combinations still fail to detect a significant percentage of stage I/II epithelial ovarian cancer. Another study (Elieser et al., 2005) measured serum concentrations of 46 biomarkers including CA-125 and amongst these, 20 proteins in combination correctly recognized more than 98% of serum samples of women with ovarian cancer compared to other benign pelvic disease. Although other malignancies were not included in this study, this multimarker panel assay provided the highest diagnostic power for early detection of ovarian cancer thus far.

Additionally, with the advent of differential gene expression analysis technologies, for example DNA microarrays and subtraction methods, many groups have now reported large collections of genes that are upregulated in epithelial ovarian cancer (United States Patent Application published under numbers; 20030124579, 20030087250, 20060014686, 20060078941, 20050095592, 20050214831, 20030219760, 20060078941, 20050214826). However, the clinical utilities with respect to ovarian cancer of one or combinations of these genes are not as yet fully determined.

There is a need for new tumor biomarkers for improving diagnosis and/or prognosis of cancer. In addition, due to the genetic diversity of tumors, and the development of chemoresistance by many patients, there exists further need for better and more universal therapeutic approaches for the treatment of cancer. Molecular targets for the development of such therapeutics may preferably show a high degree of specificity for the tumor tissues compared to other somatic tissues, which will serve to minimize or eliminate undesired side effects, and increase the efficacy of the therapeutic candidates.

This present invention tries to address these needs and other needs.

In international application No. PCT/CA2007/001134 (published on Dec. 27, 2007 under no. WO/2007/147265), the Applicant has provided several nucleic acid and polypeptide sequences that has been found to be preferentially expressed in cancer cells. The entire content of this application is herewith enclosed by reference. Here the Applicant has investigated further on one of the nucleic acid sequence disclosed in international application No. PCT/CA2007/001134 and came to the unexpected discovery that this sequence expresses a polypeptide similar to the envelope of a human endogenous retrovirus (HERV) and that this polypeptide is specifically expressed in ovarian cancer, in renal cancer and in leukemia.

### SUMMARY OF THE INVENTION

In accordance with the present invention, there is provided new polynucleotide sequences and new polypeptide sequences as well as compositions, antibodies specific for these sequences, vectors and cells comprising a recombinant form of these new sequences.

The present invention also provides methods of detecting cancer cells using single or multiple polynucleotides and/or polypeptide sequences which are specific to these tumor cells. Some of the polynucleotides and/or polypeptides sequences provided herein are differentially expressed in cancer cells compared to normal cells. These polynucleotides and/or polypeptides sequences are particularly expressed in ovarian cancer cells in comparison to normal cells found in the ovary and may also be used to distinguish between malignant ovarian cancer and an ovarian cancer of a low malignancy potential and/or a normal state (individual free of ovarian cancer).

Also encompassed by the present invention are diagnostic methods, prognostic methods, methods of detection, kits, arrays, librairies and assays which comprises one or more polypeptide and/or polynucleotide sequences or antibodies described herein as well as new therapeutic avenues for cancer treatment.

The Applicant has come to the surprising discovery that polynucleotide and/or polypeptide sequences described herein are preferentially upregulated in malignant ovarian cancer compared to low malignancy potential ovarian cancer and/or compared to normal cells.

The Applicant has also come to the surprising discovery that some of the sequences described herein are not only expressed in ovarian cancer but also in cells of renal cancer and leukemia. As such, these sequences, either alone or in combination with other sequences known to be useful cancer markers (see for example PCT/CA2007/001134) may be used in the detection of cancer cells or used in the diagnosis or prognosis of cancer. Therefore, some sequences described herein not only find utility in the field of ovarian cancer detection and treatment but also in the detection and treatment of other types of cancer.

Using sequences of the present invention, one may readily identify a cell as being cancerous. As such sequences may be used to identify a cell as being an ovarian cancer cell, a renal cancer cell, a leukemia cell.

The sequences may further be used to treat cancer or to identify compounds useful in the treatment of cancer including, ovarian cancer (i.e, LMP and/or malignant ovarian cancer), renal cancer or leukemia.

### BRIEF DESCRIPTION OF THE DRAWINGS

In the appended drawings:

FIG. 1 is a picture of RT-PCR results showing the differential expression data for STAR selected ovarian cancer-related human sequences. Complementary DNAs were prepared using random hexamers from RAMP amplified RNA from six human LMP samples and at least twenty malignant ovarian tumor samples (Table B) as indicated in the figures. The cDNAs were quantified and used as templates for PCR with gene-specific primers using standard methods known to those skilled in the art.

A primer pair, OGS 1212 (AAGCATAGCCATAGGTGATTGG; SEQ. ID. NO. 20) and OGS 1213 (ACAGGTATCAGACAAGGGAGCAG; SEQ. ID. NO. 21) for SEQ. ID. NO. 1 was used to perform RT-PCR on LMP samples, different stages/grades of ovarian cancer and normal human tissue samples. As indicated by the expected PCR amplicon product (indicated as AB-0532), increased expression of SEQ. ID. NO. 1 mRNA was evident in a large majority of the ovarian cancer lanes and weaker expression was seen in LMP samples. Expression was observed in a few normal tissue samples such as kidney, thymus and spleen (lanes 14, 16 and 23, respectively). Equal amounts of template cDNA used in each PCR reaction was confirmed by reamplifying GAPDH with a specific primer pair, OGS 315 (TGAAGGTCGGAGTCAACGGATTTGGT; SEQ. ID. NO. 36) and OGS 316 (CATGTGGGCCATGAGGTCCACCAC; SEQ. ID. NO. 37) for this housekeeping gene. These results confirm the upregulation of the gene expression for SEQ. ID. NO. 1 in malignant ovarian cancer;

FIG. 2 is a picture of RT-PCR data showing the differential expression data for the STAR selected ovarian cancer-related human SEQ. ID. NO. 1 in RNA samples derived from the NCI-60 panel of cancer cell lines. A primer pair, OGS 1212 (AAGCATAGCCATAGGTGATTGG; SEQ. ID. NO. 20) and OGS 1213 (ACAGGTATCAGACAAGGGAGCAG; SEQ. ID. NO. 21) for SEQ. ID. NO. 1 was used to perform RT-PCR. As indicated by the expected PCR amplicon, increased expression of SEQ. ID. NO. 1 mRNA was evident only in ovarian and renal cancer and leukemia;

FIG. 3A is a picture of the macroarray hybridization results showing the differential expression data for STAR selected ovarian cancer-related human SEQ. ID. NO. 4. The STAR dsDNA clone representing SEQ. ID. NO. 4 was labeled with ³²P and hybridized to the macroarray. The hybridization results obtained confirm its upregulation in a majority of malignant ovarian cancer samples (A-F 2 and A-G 3-4) compared to LMP samples (A-F 1). Significant expression of this sequence was also evident in the seven (adrenal (A7), breast (B7), trachea (D7), placenta (F7), lung (A8), kidney (F8) and fallopian tube (F9)) of the 30 normal tissues;

FIG. 3B is a picture of RT-PCR data showing the differential expression data for the STAR selected ovarian cancer-related human SEQ. ID. NO. 4 in RNA samples derived from the NCI-60 panel of cancer cell lines. A primer pair, OGS 1035 (CTGCCTGCCAACCTTTCCATTTCT; SEQ. ID. NO. 18) and OGS 1036 (TGAGCAGCCACAGCAGCATTAGG; SEQ. ID. NO. 19) for SEQ. ID. NO. 4 was used to perform RT-PCR. As indicated by the expected PCR amplicon, increased expression of SEQ. ID. NO. 4 mRNA was evident in all cancer types but weak in CNS cancer and leukemia.

FIG. 4A represents the open reading frame identified at position 1425 to 1859 (encoding a polypeptide identified herein as SEQ ID NO:3) of the sense strand of SEQ ID NO.:2 using the program “ORF finder” available at http://www.ncbi.nlm.nih.gov/projects/gorf/;

FIG. 4B represents the open reading frame identified at position 518 to 820 (encoding a polypeptide identified herein as SEQ ID NO:22) of the sense strand of SEQ ID NO.:2 using the same program as for FIG. 4A;

FIG. 4C represents the open reading frame identified at position 112 to 339 (encoding a polypeptide identified herein as SEQ ID NO:23) of the sense strand of SEQ ID NO.:2 using the same program as for FIG. 4A;

FIG. 4D represents the open reading frame identified at position 3 to 179 (encoding a polypeptide identified herein as SEQ ID NO:24) of the sense strand of SEQ ID NO.:2 using the same program as for FIG. 4A;

FIG. 4E represents the open reading frame identified at position 1021 to 1218 (encoding a polypeptide identified herein as SEQ ID NO:25) of the sense strand of SEQ ID NO.:2 using the same program as for FIG. 4A;

FIG. 4F represents the open reading frame identified at position 1336 to 1461 (encoding a polypeptide identified herein as SEQ ID NO:26) of the sense strand of SEQ ID NO.:2 using the same program as for FIG. 4A;

FIG. 5A represents the open reading frame identified at position 120 to 410 (encoding a polypeptide identified herein as SEQ ID NO:27) of the anti-sense strand of SEQ ID NO.:2 using the same program as for FIG. 4A;

FIG. 5B represents the open reading frame identified at position 427 to 639 (encoding a polypeptide identified herein as SEQ ID NO:28) of the anti-sense strand of SEQ ID NO.:2 using the same program as for FIG. 4A

FIG. 5C represents the open reading frame identified at position 1228 to 1401 (encoding a polypeptide identified herein as SEQ ID NO:29) of the anti-sense strand of SEQ ID NO.:2 using the same program as for FIG. 4A;

FIG. 5D represents the open reading frame identified at position 828 to 980 (encoding a polypeptide identified herein as SEQ ID NO:30) of the anti-sense strand of SEQ ID NO.:2 using the same program as for FIG. 4A;

FIG. 5E represents the open reading frame identified at position 1196 to 1318 (encoding a polypeptide identified herein as SEQ ID NO:31) of the anti-sense strand of SEQ ID NO.:2 using the same program as for FIG. 4A;

FIG. 6 is an histogram illustrating the results of fluorescence-activated cell sorting using Fabs generated against SEQ ID NO:3, and;

FIG. 7 illustrates the results of immunohistochemistry performed with A) antibody 1561, B) antibody 1621 and C) antibody 1771.

### DETAILED DESCRIPTION OF THE INVENTION

### Differentially Expressed Nucleic Acid Sequences

The present invention relates in one aspect thereof to nucleic acid sequences which are differentially expressed in cancer cells compared to normal cells. These nucleic acid sequences may be used in the detection and treatment of cancer.

The term “NSEQ” as used herein includes polynucleotides sequences comprising or consisting of the nucleic acid sequences (e.g., SEQ ID NO.:2) described herein (e.g., an isolated form) or comprising or consisting of a fragment of the nucleic acid sequences described herein. The term “NSEQ” additionally includes a sequence substantially identical to any one of the above. The term “NSEQ” also includes a polynucleotide sequence able to encode any one of the polypeptides described herein or a polypeptide fragment of any one of the above. Finally, the term “NSEQ” includes a sequence substantially complementary to any one of the above. It is to be understood herein that the term “NSEQ” may include or may exclude SEQ ID NO.:1.

The term “inhibitory NSEQ” may generally refer in some instances to a sequence substantially complementary to SEQ ID NO.:2, substantially complementary to a fragment of SEQ ID NO:2, substantially complementary to a sequence substantially identical to SEQ ID NO:2: and which may have attenuating or even inhibitory action againts the transcription of a mRNA or against expression of a polypeptide encoded by SEQ ID NO:2. Suitable “inhibitory NSEQ” may inlude, for example, siRNAs and may have for example and without limitation from about 10 to about 30 nucleotides, from about 10 to about 25 nucleotides or from about 15 to about 20 nucleotides. It is to be understood herein that the ter, “inhibitory NSEQ” may include or may exclude those nucleic acid sequences derived from SEQ ID NO.:1 (e.g., sequence substantially complementary to SEQ ID NO.:1, substantially complementary to a fragment of SEQ ID NO:1, substantially complementary to a sequence substantially identical to SEQ ID NO:1: and which may have attenuating or even inhibitory action againts the transcription of a mRNA or against expression of a polypeptide encoded by SEQ ID NO:1).

Exemplary fragments of SEQ ID NO:2 fragments that are encompassed by the present invention include fragments of from 10 to 2021 nucleotides long that are comprised and included within SEQ ID NO:2 (either in the coding or non-coding region) or within SEQ ID NO:2 complement. Yet other exemplary fragments include those comprised and included within SEQ ID NO:2 with the exclusion of SEQ ID NO:1, SEQ ID NO:1 fragments and/or complement thereof. Fragments of SEQ ID NO:2 especially encompassed by the present invention are those covering an open reading frame encoding a polypeptide.

As used herein the term “identity”, “sequence identity” or “identical” in the context of nucleic acids relates to (consecutive) nucleotides of a nucleotide sequence with reference to an original nucleotide sequence which when compared are the same or have a specified percentage of nucleotides which are the same.

The identity may be compared over a portion or over the total sequence of a nucleic acid sequence. Thus, “identity” may be compared, for example, over a portion of the nucleic acid 10, 19, 20, 25, 30, 50, 100 nucleotides or more (and any number therebetween) or even over the entire length of a polynucleotide sequence described herein. It is to be understood herein that gaps of non-identical nucleotides may be found between identical nucleic acids regions (identical nucleotides). For example, a polynucleotide may have 100% identity with another polynucleotide over a portion thereof. However, when the entire sequence of both polynucleotides is compared, the two polynucleotides may have 50% of their overall (total) sequence identity to one another.

Percent identity may be determined, for example, with n algorithm GAP, BESTFIT, or FASTA in the Wisconsin Genetics Software Package Release 7.0, using default gap weights.

Polynucleotides of the present invention or portion thereof having from about 50 to about 100% and any individual range therebetween, such as about 60 to about 100% or about 70 to about 100% or about 80 to about 100% or about 85% to about 100%, about 90% to about 100%, about 95% to about 100% sequence identity with an original polynucleotide are encompassed herewith. It is known by those of skill in the art, that a polynucleotide having from about 50% to 100% identity may function (e.g., anneal to a substantially complementary sequence) in a manner similar to an original polynucleotide and therefore may be used in replacement of an original polynucleotide. For example a polynucleotide (a nucleic acid sequence) may comprise or have from about 50% to about 100% identity with an original polynucleotide over a defined region and may still work as efficiently or sufficiently to achieve the present invention.

The term “substantially identical” used to define the polynucleotides of the present invention refers to polynucleotides which have, for example, from 50% to 100% sequence identity and any range therebetween but preferably at least 80%, at least 85%, at least 90%, at least 95% sequence identity and also include 100% identity with that of an original sequence (including sequences 100% identical over the entire length of the polynucleotide sequence). Substantially identical sequence that are encompassed by the present invention are those having the percentage identity mentioned above where the percentage of identity is determined over at least 20 nucleotides of the polynucleotide sequence.

“Substantially identical” polynucleotide sequences may be identified by providing a probe of about 10 to about 25, or more or about 10 to about 20 nucleotides long (or longer) based on the sequence of and complementary sequence thereof and hybridizing a library of polynucleotide (e.g., cDNA or else) originating from another species, tissue, cell, individual etc. A polynucleotide which hybridizes under highly stringent conditions (e.g., 6×SCC, 65° C.) to the probe may be isolated and identified using methods known in the art. A sequence “substantially identical” includes for example, an isolated allelic variant, an isolated splice variant, an isolated non-human ortholog, a modified NSEQ etc.

As used herein the terms “sequence complementarity” refers to (consecutive) nucleotides of a nucleotide sequence which are complementary to a reference (original) nucleotide sequence. The complementarity may be compared over a region or over the total sequence of a nucleic acid sequence.

Polynucleotides of the present invention or portion thereof having from about 50 to about 100%, or about 60 to about 100% or about 70 to about 100% or about 80 to about 100% or about 85%, about 90%, about 95% to about 100% sequence complementarity with an original polynucleotide are thus encompassed herewith. It is known by those of skill in the art, that a polynucleotide having from about 50% to 100% complementarity with an original sequence may anneal to that sequence in a manner sufficient to carry out the present invention (e.g., inhibit expression of the original polynucleotide).

The term “substantially complementary” used to define the polynucleotides of the present invention refers to polynucleotides which have, for example, from 50% to 100% sequence complementarity and any range therebetween but preferably at least 80%, at least 85%, at least 90%, at least 95% sequence complementarity and also include 100% complementarity with that of an original sequence (including sequences 100% complementarity over the entire length of the polynucleotide sequence). Substantially complementary sequence that are encompassed by the present invention are those having the percentage complementarity mentioned above where the percentage of complementarity is determined over at least 20 nucleotides of the polynucleotide sequence.

As used herein the terms “polynucleotide” or “nucleic acids” are used interchangeably and generally refers to any polyribonucleotide or polydeoxyribo-nucleotide, which may be unmodified RNA or DNA, or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated, methylated bases and unusual bases such as inosine. A variety of modifications may be made to DNA and RNA; thus “polynucleotide” embraces chemically, enzymatically or metabolically modified forms of polynucleotides as typically found or not in nature, as well as the chemical forms of DNA and RNA characteristic of viruses and cells. “Polynucleotide” includes but is not limited to linear and end-closed molecules. “Polynucleotide” also embraces relatively short polynucleotides, often referred to as oligonucleotides.

Unless specifically mentioned herein, the term “nucleotide” is used generically and encompasses nucleotides, nucleosides as well as their modified form (O-methyl, etc.).

Exemplary embodiments of nucleic acid of the present invention includes an isolated polynucleotide (e.g., exogenous form of) which may comprise a member selected from the group consisting of;
a) A nucleic acid which may comprise or consist in SEQ ID NO:2;
b) A nucleic acid comprising a complement of SEQ ID NO:2,
c) A nucleic acid at least 90%, 91%, 92%, 93%, 94%, 95% or more identical to SEQ ID NO:2 or to a complement thereof and;
d) a nucleic acid comprising a fragment of a), b) or c);

Nucleic acids which are encompassed by the present invention are those which are at least 90%, 91%, 92%, 93%, 94%, 95% or more identical to SEQ ID NO:2 or to a complement thereof and having a length comprised between (inclusively) 20 nucleotides and the total length of SEQ ID NO:2.

In accordance with the present invention, the percentage of identity may be determined over a fragment of SEQ ID NO:2.

Further in accordance with the present invention, the percentage of identity may be determined over the entire length of SEQ ID NO:2.

Exemplary embodiments of nucleic acids are those having at least 92%, 93%, 94%, 95%, 96%, 97%, 98%, 99% or 100% identity over a fragment which comprises nucleotides 1425 to 1856 of SEQ ID NO:2.

Exemplary and non-limitative embodiments of nucleic acid fragments include the following
a) a fragment which may comprise nucleotides 1425 to 1856 of SEQ ID NO:2 or a complement thereof;
b) a fragment which may comprise nucleotides 518 to 817 of SEQ ID NO:2 or a complement thereof;
c) a fragment which may comprise nucleotides 112 to 336 of SEQ ID NO:2 or a complement thereof; or
d) a fragment which may comprise nucleotides 3 to 176 of SEQ ID NO:2 or a complement thereof.

Other exemplary and non-limitative embodiments of nucleic acid fragments include the following
a) a fragment which may comprise nucleotides 1425 to 1859 of SEQ ID NO:2 or a complement thereof;
b) a fragment which may comprise nucleotides 518 to 820 of SEQ ID NO:2 or a complement thereof;
c) a fragment which may comprise nucleotides 112 to 339 of SEQ ID NO:2 or a complement thereof; or
d) a fragment which may comprise nucleotides 3 to 179 of SEQ ID NO:2 or a complement thereof.

Yet other exemplary and non-limitative embodiments of nucleic acid fragments include:
a) a fragment of from 10 to 434 (consecutive) nucleotides located between nucleotide 1425 and 1859 (inclusively) of SEQ ID NO:2 or a complement thereof
b) a fragment of from 10 to 302 (consecutive) nucleotides located between nucleotide 518 and 820 (inclusively) of SEQ ID NO:2 or a complement thereof;
c) a fragment of from 10 to 227 (consecutive) nucleotides located between nucleotide 112 and 339 (inclusively) of SEQ ID NO:2 or a complement thereof; or
d) a fragment of from 10 to 116 (consecutive) nucleotides located between nucleotide 3 and 179 (inclusively) of SEQ ID NO:2 or a complement thereof.

Some aspects of the invention relates to polynucleotides that includes SEQ ID NO:1, SEQ ID NO:1 fragments and complement thereof, while other aspects of the invention may exclude SEQ ID NO:1 or SEQ ID NO:1 fragments (e.g., a fragments of 10 to 282 nucleotides found within SEQ ID NO:1) or complement thereof.

As such the present invention relates to an isolated nucleic acid which may be selected from the group consisting of;
a) A nucleic acid comprising or consisting of SEQ ID NO:2;
b) A nucleic acid comprising a complement of SEQ ID NO:2,
c) A nucleic acid at least 90% identical to SEQ ID NO:2 or a complement thereof and;
d) a nucleic acid comprising a fragment of a), b) or c);
   provided that the nucleic acid is not SEQ ID.:1.

It is to be understood herein that the term “fragment” with respect to nucleic acids encompasses nucleic acids which are smaller than the original reference nucleic acid by at least one nucleotide. A fragment may be as short as 10, 11, 12, 13, 14, 15, 16, 17, 18, 19, 20, 21, 22, 23, 24, 25 etc. nucleotides. For example, the term “a fragment of from 10 to 434 nucleotides”, encompasses a fragment having any number of nucleotides comprised within 10 and 434 nucleotides (inclusively), such as 20, 25, 40, 56, 288, 359, 411, 434 nucleotides and any integer number found between 10 and 434. The same applies to any similar terms found herein.

It is also to be understood herein that a nucleic acid may comprise a portion corresponding to a nucleic acid fragment of the present invention and another unrelated nucleic acid portion.

Vectors (e.g., a viral vector, a mammalian vector, a plasmid, a cosmid, etc.) which may comprise the polynucleotides described herein are also encompassed by the present invention. The vector may be, for example, an expression vector.

The present invention also provides a library of polynucleotide comprising at least one polynucleotide (e.g., at least two, etc.) and which include SEQ ID NO:2, SEQ ID NO:2 fragments or complements thereof. The library may be, for example, an expression library. Some or all of the polynucleotides described herein may be contained within an expression vector. The present invention also relates to a polypeptide library which may comprise at least one (e.g., at least two, etc.) polypeptide as described herein.

In another aspect, the present invention provides arrays which may comprise at least one polynucleotide (e.g., at least two, etc.) described herein.

The present invention also provides an isolated cell (e.g., an isolated live cell such as an isolated mammalian cell, a bacterial cell, a yeast cell, an insect cell, etc.) which may comprise the polynucleotide, the vector or the polypeptide described herein.

In yet a further aspect the present invention relates to a composition comprising the polynucleotide and/or polypeptide described herein.

In accordance with the present invention, the composition may be, for example, a pharmaceutical composition which may comprise a polynucleotide and/or a polypeptide described herein and a pharmaceutically acceptable carrier. More specifically, the pharmaceutical composition may be used for the treatment of ovarian cancer and/or for inhibiting the growth of an ovarian cancer cell.

These above sequences may represent powerful markers of cancer and more particularly of, ovarian cancer, leukemia, or renal cancer.

Based on the results presented herein and upon reading the present description, a person skilled in the art will understand that the emission of a positive signal upon testing (hybridization, PCR amplification etc.) for the presence of a given sequence amongst those expressed in a cancer cell, indicates that such sequence is specifically expressed in that type of cancer cell. A person skilled in the art will also understand that, sequences which are specifically expressed in a certain types of cancer cell may be used for developing tools for the detection of this specific type of cancer cell and may also be used as targets in the development of anticancer drugs.

A positive signal may be in the form of a band in a gel following electrophoresis, Northern blot or Western blot, a PCR fragment detected by emission of fluorescence, colorimetry etc.

As it will be understood, sequences which are particularly useful for the development of tools for the detection of cancer cells may preferably be expressed at lower levels in at least some normal cells (non-cancerous cells).

Therapeutic uses and methods are also encompassed herewith.

The invention therefore provides polynucleotides which may be able to lower or inhibit the growth of an ovarian cancer cell (e.g., in a mammal or mammalian cell thereof).

The present invention also relates in a further aspect to the use of a polynucleotide sequence described herein for reducing, lowering or inhibiting the growth of a cancer cell. More particularly, the present invention relates to the use of a nucleic acid described herein in the treatment, detection or diagnosis of cancer (e.g., ovarian cancer, renal cancer, leukemia).

The present invention further encompasses immunizing an individual by administering a NSEQ (e.g., in an expression vector, naked DNA, etc.) or a PSEQ.

The present invention also relates to a method of reducing, lowering or slowing the growth of an ovarian cancer cell in an individual in need thereof. The method may comprise administering to the individual a polynucleotide sequence which may be selected from the group consisting of a complement of SEQ ID NO:2 or of a SEQ ID NO:2 fragment. More particularly, the present invention relates to a methods of treating, detecting or diagnosing cancer (e.g., ovarian cancer, renal cancer, leukemia) in an individual in need.

In some embodiments the nucleic acid will be those which are derived from SEQ ID NO:2 (e.g., complement, fragments, susbtantially identical, substantially complementary). In other embodiments the nucleic acid will be those which are derived from SEQ ID NO:2 (e.g., complement, fragments, susbtantially identical, substantially complementary) provided that these nucleic acids are not derived from SEQ ID NO:1 (e.g., including complement, fragments, susbtantially identical, substantially complementary).

The present invention therefore provides in yet another aspect thereof, a siRNA or shRNA molecule that is able to lower the expression of SEQ ID NO:2 or of a SEQ ID NO:2 fragment. In some embodiments the siRNA or shRNA may be derived from any region of SEQ ID NO:2 or SEQ ID NO:2 complement while in other embodiments, the siRNA or shRNA may be derived from a region of SEQ ID NO:2 or SEQ ID NO:2 complement located outside of the region corresponding to SEQ ID NO:1 or SEQ ID NO:1 complement. The siRNA or shRNA may be provided in compositions including a buffer, saline or water. The siRNa or shRNA may also be provided in pharmaceutical compositions comprising a pharmaceutically acceptable carrier.

The present invention also provides a kit for the diagnosis of cancer. The kit may comprise at least one polynucleotide as described herein and/or a reagent capable of specifically binding at least one polynucleotide described herein.

In a further aspect, the present invention relates to an isolated polypeptide encoded by the polynucleotide described herein.

More particularly, the present invention relates to a polypeptide which is preferentially expressed in ovarian cancer cells in comparison with normal ovarian cells, the polypeptide may comprise an amino acid sequence encoded by SEQ ID NO:2.

### Differentially Expressed Polypeptide Sequences

The present invention relates in another aspect thereof to polypeptide sequences. More particularly polypeptide sequences which are differentially expressed in cancer cells compared to normal cells are encompassed herewith. These polypeptide sequences may be used in the detection, diagnosis and treatment of cancer.

As used herein the term “PSEQ” refers generally to each and every polypeptide sequences described herein such as, for example, any polypeptide sequences encoded (putatively encoded) by any one of NSEQ described herein including their isolated or substantially purified form, fragments of the polypeptides described herein or larger amino acid sequence comprising the polypeptides or fragments as well as variants described herein.

“Polypeptides” refers to any peptide or protein comprising two or more amino acids joined to each other by peptide bonds or modified peptide bonds (i.e., peptide isosteres). “Polypeptide” refers to both short chains, commonly referred as peptides, oligopeptides or oligomers, and to longer chains generally referred to as proteins. As described above, polypeptides may contain amino acids other than the 20 gene-encoded amino acids.

As used herein the term “polypeptide variant” or “variant” relates to mutants, chimeras, fusions, a polypeptide comprising at least one amino acid deletion, a polypeptide comprising at least one amino acid insertion or addition, a polypeptide comprising at least one amino acid substitutions, and any other type of modifications made relative to a given polypeptide.

Generally, the degree of similarity and identity between polypeptide sequence may been determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity will therefore be indicative of amino acids which are identical in comparison with the original peptide and which may occupy the same or similar position.

Percent similarity will be indicative of amino acids which are identical and those which are replaced with conservative amino acid substitution in comparison with the original peptide at the same or similar position.

A “variant” is thus to be understood herein as a molecule having a biological activity and/or chemical structure similar to that of a polypeptide described herein. A “variant” may have sequence similarity with that of an original sequence or a portion of an original sequence and may also have a modification of its structure as discussed herein. For example, a “variant” may have at least 80% or 85% or 90% sequence similarity with an original sequence or a portion of an original sequence. A “variant” may also have, for example; at least 70% or even 50% sequence similarity with an original sequence or a portion of an original sequence and may function in a suitable manner.

A “derivative” is to be understood herein as a polypeptide originating from an original sequence or from a portion of an original sequence and which may comprise one or more modification; for example, one or more modification in the amino acid sequence (e.g., an amino acid addition, deletion, insertion, substitution etc.), one or more modification in the backbone or side-chain of one or more amino acid, or an addition of a group or another molecule to one or more amino acids (side-chains or backbone). Biologically active derivatives of the carrier described herein are encompassed by the present invention. Also, an “derivative” include polypeptides described herein and variants havingone or more modification in a backbone or side-chain of an amino acid, or an addition of a group or another molecule (alkylation, pegylation, etc.).

As used herein the term “biologically active” refers to a variant which retains some or all of the biological activity of the original polypeptide, i.e., to have some of the activity or function associated with the polypeptide described herein, or to be able to promote or inhibit the growth of cancer cells.

Therefore, any polypeptide having a modification compared to an original polypeptide which does not destroy significantly a desired activity, function or immunogenicity is encompassed herein. It is well known in the art, that a number of modifications may be made to the polypeptides of the present invention without deleteriously affecting their biological activity. These modifications may, on the other hand, keep or increase the biological activity of the original polypeptide or may optimize one or more of the particularity (e.g. stability, bioavailability, etc.) of the polypeptides of the present invention which, in some instance might be desirable. Polypeptides of the present invention may comprise for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino- or carboxy-terminus. It will be appreciated that the same type of modification may be present in the same or varying degrees at several sites in a given polypeptide. Also, a given polypeptide may contain many types of modifications. It is to be understood herein that more than one modification to the polypeptides described herein are encompassed by the present invention to the extent that the biological activity is similar to the original (parent) polypeptide.

As discussed above, polypeptide modification may comprise, for example, amino acid insertion, deletion and substitution (i.e., replacement), either conservative or non-conservative (e.g., D-amino acids, desamino acids) in the polypeptide sequence where such changes do not substantially alter the overall biological activity of the polypeptide.

Example of substitutions may be those, which are conservative (i.e., wherein a residue is replaced by another of the same general type or group) or when wanted, non-conservative (i.e., wherein a residue is replaced by an amino acid of another type). In addition, a non-naturally occurring amino acid may substitute for a naturally occurring amino acid (i.e., non-naturally occurring conservative amino acid substitution or a non-naturally occurring non-conservative amino acid substitution).

It should noted that if the polypeptides are made synthetically, substitutions by amino acids, which are not naturally encoded by DNA (non-naturally occurring or unnatural amino acid) may also be made.

A non-naturally occurring amino acid is to be understood herein as an amino acid which is not naturally produced or found in a mammal. A non-naturally occurring amino acid comprises a D-amino acid, an amino acid having an acetylaminomethyl group attached to a sulfur atom of a cysteine, a pegylated amino acid, etc. The inclusion of a non-naturally occurring amino acid in a defined polypeptide sequence will therefore generate a derivative of the original polypeptide. Non-naturally occurring amino acids (residues) include also the omega amino acids of the formula NH₂(CH₂)ₙCOOH wherein n is 2-6, neutral nonpolar amino acids, such as sarcosine, t-butyl alanine, t-butyl glycine, N-methyl isoleucine, norleucine, etc. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that variants may be generated by substitutional mutagenesis and retain the biological activity of the polypeptides of the present invention. These variants have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr)
(group 3) acidic: Aspartic acid (Asp), Glutamic acid (Glu)
(group 4) basic: Asparagine (Asn), Glutamine (Gln), Histidine (His), Lysine (Lys), Arginine (Arg)
(group 5) residues that influence chain orientation: Glycine (Gly), Proline
(Pro); and (group 6) aromatic: Tryptophan (Trp), Tyrosine (Tyr), Phenylalanine (Phe)

Non-conservative substitutions will entail exchanging a member of one of these classes for another.

TABLE 1
Examplary amino acid substitution

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg, Asp | Gln |
| Asp (D) | Glu, Asn | Glu |
| Cys (C) | Ser, Ala | Ser |
| Gln (Q) | Asn; Glu | Asn |
| Glu (E) | Asp, Gln | Asp |
| Gly (G) | Ala | Ala |
| His (H) | Asn, Gln, Lys, Arg, | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | norleucine | |

Exemplary embodiments of polypeptides of the present invention thus includes the polypeptides encoded by SEQ ID NO.:2 as well as fragments and variants.

Some aspects of the invention related to polypeptides, compositions, kits and their method of use may include SEQ ID NO:32, SEQ ID NO:33 or polypeptides encoded by SEQ ID NO:1, while other aspects of the invention may exclude SEQ ID NO:32, SEQ ID NO:33 or those encoded by SEQ ID NO:1.

Exemplary and non-limitative embodiments of the invention, include an isolated polypeptide which may be selected from the group consisting of;
a) A polypeptide which may comprise a sequence at least 75% identical to SEQ ID NO:3;
b) A polypeptide which may comprise a sequence at least 75% identical to SEQ ID NO:22;
c) A polypeptide which may comprise a sequence at least 75% identical to SEQ ID NO:23, and;
d) A polypeptide which may comprise a sequence at least 75% identical to SEQ ID NO:24.

More particular embodiment of the invention includes polypeptide which comprises a sequence at least 80% identical, at least 85% identical, at least 90% identical, at least 95% identical or at least 100% identical to SEQ ID NO:3, SEQ ID NO:22, SEQ ID NO:23 or SEQ ID NO:24.

Specific embodiment of the invention includes polypeptide which consists in SEQ ID NO:3, SEQ ID NO:22, SEQ ID NO:23 or SEQ ID NO:24.

In accordance with the present invention, the identity of the corresponding polypeptide may determined over 20 consecutive amino acids or more including the total length of SEQ ID NO:3, SEQ ID NO:22, SEQ ID NO:23 or SEQ ID NO:24.

Further in accordance with the present invention, the identity of the corresponding polypeptide may determined over the entire length of SEQ ID NO:3, SEQ ID NO:22, SEQ ID NO:23 or SEQ ID NO:24.

Other exemplary and non-limitative embodiments of the invention, includes polypeptide which may be selected from the group consisting of;
a) A polypeptide which may comprise or consist in a fragment of from 6 to 143 consecutive amino acids of SEQ ID NO:3;
b) A polypeptide which may comprise or consist in a fragment of from 6 to 99 consecutive amino acids of SEQ ID NO:22;
c) A polypeptide which may comprise or consist in a fragment of from 6 to 74 consecutive amino acids of SEQ ID NO:23, and;
d) A polypeptide which may comprise or consist in a fragment of from 6 to 57 consecutive amino acids of SEQ ID NO:24.

These fragments may be used for example in compositions for immunizing animals for generating antibodies. It is to be understood herein that these fragments may be fused with other foreign polypeptides (e.g., KHL, BSA) for immunization purposes or may be comprised within a larger fragment derived from its corresponding sequence (SEQ ID NO:3, SEQ ID NO:22, SEQ ID NO:23 or SEQ ID NO:24).

It is to be understood herein that the term “fragment” with respect to polypeptides encompasses polypeptides which are smaller than the original reference polypeptide by at least one amino acid. A fragment may be as short as 6, 7, 8, 9, 10, 11, 12, 13, 14, 15, 16, 17, 18, 19, 20, etc. amino acids. For example, the term “a fragment of from 6 to 143 amino acids” encompasses a fragment having any number of amino acids comprised within 6 to 143 amino acids (inclusively), such as 20, 25, 33, 111, 142 and any integer number found between 6 to 143. The same applies to any similar terms found herein.

It is also to be understood herein that a polypeptide may comprise a portion corresponding to a polypeptide fragment of the present invention and another unrelated polypeptide portion.

In accordance with the present invention, a variant may comprise, for example, at least one amino acid substitution, deletion or insertion in its amino acid sequence.

The substitution may thus be conservative or non-conservative. The polypeptide variant may be a biologically active variant or an immunogenic variant which may comprise, for example, at least one amino acid substitution (conservative or non conservative), for example, 1 to 5, 1 to 10, 1 to 15, 1 to 20, 1 to 50 etc. (including any number there between) compared to the original sequence. An immunogenic variant may comprise, for example, at least one amino acid substitution compared to the original sequence and may still be recognized by an antibody or antigen binding fragment specific for the original sequence.

In accordance with the present invention, a polypeptide fragment may comprise, for example, at least 6 consecutive amino acids, at least 7 consecutive amino acids, at least 8 consecutive amino acids or more of an amino acids (up to the total length of the polypeptide) selected from the group consisting of polypeptides encoded by SEQ ID NO:2 (SEQ ID NO:3, SEQ ID NO:22, SEQ ID NO:23 or SEQ ID NO:24), including variants thereof. The fragment may be immunogenic and may be used for the purpose, for example, of generating antibodies.

Variants of the present invention therefore comprise those which may have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with an original sequence or a portion of an original sequence.

Exemplary embodiments of variants are those having at least 75% sequence identity to a sequence described herein and 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence similarity with an original sequence or a portion of an original sequence.

For a purpose of concision the applicant provides herein Table 2 illustrating exemplary embodiments of individual variants encompassed by the present invention and comprising the specified % sequence identity and % sequence similarity. Each “X” is to be construed as defining a given variant.

TABLE 2

| | Percent (%) sequence identity | | | | | | | | | | | | | | | | | | | | | | | | | | |
|---|---|---|---|---|---|---|---|---|---|---|---|---|---|---|---|---|---|---|---|---|---|---|---|---|---|---|---|
| Percent | | 75 | 76 | 77 | 78 | 79 | 80 | 81 | 82 | 83 | 84 | 85 | 86 | 87 | 88 | 89 | 90 | 91 | 92 | 93 | 94 | 95 | 96 | 97 | 98 | 99 | 100 |
| (%) | 75 | X | | | | | | | | | | | | | | | | | | | | | | | | | |
| sequence | 76 | X | X | | | | | | | | | | | | | | | | | | | | | | | | |
| similarity | 77 | X | X | X | | | | | | | | | | | | | | | | | | | | | | | |
| | 78 | X | X | X | X | | | | | | | | | | | | | | | | | | | | | | |
| | 79 | X | X | X | X | X | | | | | | | | | | | | | | | | | | | | | |
| | 80 | X | X | X | X | X | X | | | | | | | | | | | | | | | | | | | | |
| | 81 | X | X | X | X | X | X | X | | | | | | | | | | | | | | | | | | | |
| | 82 | X | X | X | X | X | X | X | X | | | | | | | | | | | | | | | | | | |
| | 83 | X | X | X | X | X | X | X | X | X | | | | | | | | | | | | | | | | | |
| | 84 | X | X | X | X | X | X | X | X | X | X | | | | | | | | | | | | | | | | |
| | 85 | X | X | X | X | X | X | X | X | X | X | X | | | | | | | | | | | | | | | |
| | 86 | X | X | X | X | X | X | X | X | X | X | X | X | | | | | | | | | | | | | | |
| | 87 | X | X | X | X | X | X | X | X | X | X | X | X | X | | | | | | | | | | | | | |
| | 88 | X | X | X | X | X | X | X | X | X | X | X | X | X | X | | | | | | | | | | | | |
| | 89 | X | X | X | X | X | X | X | X | X | X | X | X | X | X | X | | | | | | | | | | | |
| | 90 | X | X | X | X | X | X | X | X | X | X | X | X | X | X | X | X | | | | | | | | | | |
| | 91 | X | X | X | X | X | X | X | X | X | X | X | X | X | X | X | X | X | | | | | | | | | |
| | 92 | X | X | X | X | X | X | X | X | X | X | X | X | X | X | X | X | X | X | | | | | | | | |
| | 93 | X | X | X | X | X | X | X | X | X | X | X | X | X | X | X | X | X | X | X | | | | | | | |
| | 94 | X | X | X | X | X | X | X | X | X | X | X | X | X | X | X | X | X | X | X | X | | | | | | |
| | 95 | X | X | X | X | X | X | X | X | X | X | X | X | X | X | X | X | X | X | X | X | X | | | | | |
| | 96 | X | X | X | X | X | X | X | X | X | X | X | X | X | X | X | X | X | X | X | X | X | X | | | | |
| | 97 | X | X | X | X | X | X | X | X | X | X | X | X | X | X | X | X | X | X | X | X | X | X | X | | | |
| | 98 | X | X | X | X | X | X | X | X | X | X | X | X | X | X | X | X | X | X | X | X | X | X | X | X | | |
| | 99 | X | X | X | X | X | X | X | X | X | X | X | X | X | X | X | X | X | X | X | X | X | X | X | X | X | |
| | 100 | X | X | X | X | X | X | X | X | X | X | X | X | X | X | X | X | X | X | X | X | X | X | X | X | X | X |

In a further aspect the present invention relates to a polypeptide which may be encoded by the isolated nucleic acids of the present invention. The present invention as well relates to the polypeptide encoded by the non-human ortholog polynucleotide, variants, derivatives and fragments thereof.

Other aspects of the invention relate to compositions comprising the polypeptides described herein, such as pharmaceutical compositions.

Yet other aspects relate to isolated cells comprising or expressing the polypeptide of the present invention.

Additional aspects relate to kits comprising the polypeptides of the present invention.

Methods of treating cancer which comprise administering one or more of the polypeptide or pharmaceutical compositions comprising one or more of the polypeptide described herein are also encompassed by the present invention.

As one skill in the art will understand, compositions which comprises a polypeptide may be used, for example, for generating antibodies against the particular polypeptide, may be used as a reference for assays and kits, etc.

### Antibodies Against Differentially Expressed Polypeptide Sequences

Antibodies (e.g., isolated antibody) and antigen binding fragments that may specifically bind to a protein or polypeptide described herein (a PSEQ) as well as nucleic acids encoding such antibodies are also encompassed by the present invention.

As used herein the term “antibody” means a monoclonal antibody, a polyclonal antibody, a single chain antibody, a chimeric antibody, a humanized antibody, a deimmunized antibody.

The term “antigen-binding fragment” encompasses fragments that are involved in the binding of the antigen, such as light chain and/or heavy chain, light chain variable region and/or heavy chain variable region an Fab fragment; an F(ab′)₂ fragment, and Fv fragment; CDRs (from the light chain and/or heavy chain), or a single-chain antibody comprising an antigen-binding fragment (e.g., a single chain Fv).

The or antigen binding fragment may originate for example, from a mouse, rat or any other mammal or from other sources such as through recombinant DNA technologies.

The antibody or antigen binding fragment derived therefrom may also be a human antibody which may be obtained, for example, from a transgenic non-human mammal capable of expressing human Ig genes. The antibody or antigen binding fragment derived therefrom may also be a humanised antibody which may comprise, for example, one or more complementarity determining regions of non-human origin. It may also comprise a surface residue of a human antibody and/or framework regions of a human antibody and/or a human constant region. The antibody or antigen binding fragment derived therefrom may also be a chimeric antibody which may comprise, for example, variable domains of a non-human antibody and constant domains of a human antibody.

The antibody or antigen binding fragment of the present invention may be mutated and selected based on an increased affinity, solubility, stability, specificity and/or for one of a polypeptide described herein and/or based on a reduced immunogenicity in a desired host or for other desirable characteristics.

Suitable antibodies may bind to unique antigenic regions or epitopes in the polypeptides, or a portion thereof. Epitopes and antigenic regions useful for generating antibodies may be found within the proteins, polypeptides or peptides by procedures available to one of skill in the art. For example, short, unique peptide sequences may be identified in the proteins and polypeptides that have little or no homology to known amino acid sequences. Preferably the region of a protein selected to act as a peptide epitope or antigen is not entirely hydrophobic; hydrophilic regions are preferred because those regions likely constitute surface epitopes rather than internal regions of the proteins and polypeptides. These surface epitopes are more readily detected in samples tested for the presence of the proteins and polypeptides. Such antibodies may include, but are not limited to, polyclonal, monoclonal, chimeric, and single chain antibodies, Fab fragments, and fragments produced by a Fab expression library. The production of antibodies is well known to one of skill in the art and is not intended to be limited herein.

Peptides may be made by any procedure known to one of skill in the art, for example, by using in vitro translation or chemical synthesis procedures or by introducing a suitable expression vector into cells. Short peptides which provide an antigenic epitope but which by themselves are too small to induce an immune response may be conjugated to a suitable carrier. Suitable carriers and methods of linkage are well known in the art. Suitable carriers are typically large macromolecules such as proteins, polysaccharides and polymeric amino acids. Examples include serum albumins, keyhole limpet hemocyanin, ovalbumin, polylysine and the like. One of skill in the art may use available procedures and coupling reagents to link the desired peptide epitope to such a carrier. For example, coupling reagents may be used to form disulfide linkages or thioether linkages from the carrier to the peptide of interest. If the peptide lacks a disulfide group, one may be provided by the addition of a cysteine residue. Alternatively, coupling may be accomplished by activation of carboxyl groups.

The minimum size of peptides useful for obtaining antigen specific antibodies may vary widely. The minimum size must be sufficient to provide an antigenic epitope that is specific to the protein or polypeptide and may therefore include the whole polypeptide sequence. The maximum size is not critical unless it is desired to obtain antibodies to one particular epitope. For example, a large polypeptide may comprise multiple epitopes, one epitope being particularly useful and a second epitope being immunodominant, etc. Typically, antigenic peptides selected from the present proteins and polypeptides will range without limitation, from 5 to about 100 amino acids in length. More typically, however, such an antigenic peptide will be a maximum of about 50 amino acids in length, and preferably a maximum of about 30 amino acids. It is usually desirable to select a sequence of about 6, 8, 10, 12 or 15 amino acids, up to about 20 or 25 amino acids (and any number therebetween).

Amino acid sequences comprising useful epitopes may be identified in a number of ways. For example, preparing a series of short peptides that taken together span the entire protein sequence may be used to screen the entire protein sequence. One of skill in the art may routinely test a few large polypeptides for the presence of an epitope showing a desired reactivity and also test progressively smaller and overlapping fragments to identify a preferred epitope with the desired specificity and reactivity.

As mentioned herein, antigenic polypeptides and peptides are useful for the production of monoclonal and polyclonal antibodies. Antibodies to a polypeptide encoded by the polynucleotides of NSEQ, polypeptide variants or portions thereof, may be generated using methods that are well known in the art. For example, monoclonal antibodies may be prepared using any technique that provides for the production of antibody or antigen binding fragment by continuous cell lines in culture. These include, but are not limited to, the hybridoma, the human B-cell hybridoma, and the EBV-hybridoma techniques. In addition, techniques developed for the production of chimeric antibodies may be used. Alternatively, techniques described for the production of single chain antibodies may be employed. Fabs that may contain specific binding sites for a polypeptide encoded by the polynucleotides of NSEQ, or a portion thereof, may also be generated. Various immunoassays may be used to identify antibodies having the desired specificity. Numerous protocols for competitive binding or immunoradiometric assays using either polyclonal or monoclonal antibodies with established specificities are well known in the art.

To obtain polyclonal antibodies, a selected animal may be immunized with a protein or polypeptide. Serum from the animal may be collected and treated according to known procedures. Polyclonal antibodies to the protein or polypeptide of interest may then be purified by affinity chromatography. Techniques for producing polyclonal antisera are well known in the art.

Monoclonal antibodies (MAbs) may be made by one of several procedures available to one of skill in the art, for example, by fusing antibody producing cells with immortalized cells and thereby making a hybridoma. The general methodology for fusion of antibody producing B cells to an immortal cell line is well within the province of one skilled in the art. Another example is the generation of MAbs from mRNA extracted from bone marrow and spleen cells of immunized animals using combinatorial antibody library technology.

One drawback of MAbs derived from animals or from derived cell lines is that although they may be administered to a patient for diagnostic or therapeutic purposes, they are often recognized as foreign antigens by the immune system and are unsuitable for continued use. Antibodies that are not recognized as foreign antigens by the human immune system have greater potential for both diagnosis and treatment. Methods for generating human and humanized antibodies are now well known in the art.

Chimeric antibodies may be constructed in which regions of a non-human MAb are replaced by their human counterparts. A preferred chimeric antibody is one that has amino acid sequences that comprise one or more complementarity determining regions (CDRs) of a non-human Mab that binds to a polypeptide encoded by the polynucleotides of NSEQ, or a portion thereof, grafted to human framework (FW) regions. Methods for producing such antibodies are well known in the art. Amino acid residues corresponding to CDRs and FWs are known to one of average skill in the art.

A variety of methods have been developed to preserve or to enhance affinity for antigen of antibodies comprising grafted CDRs. One way is to include in the chimeric antibody the foreign framework residues that influence the conformation of the CDR regions. A second way is to graft the foreign CDRs or portion thereof onto human variable domains with the closest homology to the foreign variable region. Thus, grafting of one or more non-human CDRs onto a human antibody may also involve the substitution of amino acid residues which are adjacent to a particular CDR sequence or which are not contiguous with the CDR sequence but which are packed against the CDR in the overall antibody variable domain structure and which affect the conformation of the CDR. Humanized antibodies of the invention therefore include human antibodies which comprise one or more non-human CDRs as well as such antibodies in which additional substitutions or replacements have been made to preserve or enhance binding characteristics.

Chimeric antibodies of the invention also include antibodies that have been humanized by replacing surface-exposed residues to make the MAb appear human. Because the internal packing of amino acid residues in the vicinity of the antigen-binding site remains unchanged, affinity is preserved. Substitution of surface-exposed residues of a polypeptide encoded by the polynucleotides of NSEQ (or a portion thereof)-antibody according to the invention for the purpose of humanization does not mean substitution of CDR residues or adjacent residues that influence affinity for a polypeptide encoded by the polynucleotides of NSEQ, or a portion thereof.

Chimeric antibodies may also include antibodies where some or all non-human constant domains have been replaced with human counterparts. This approach has the advantage that the antigen-binding site remains unaffected. However, significant amounts of non-human sequences may be present where variable domains are derived entirely from non-human antibodies.

Antibodies of the invention include human antibodies that are antibodies consisting essentially of human sequences. Human antibodies may be obtained from phage display libraries wherein combinations of human heavy and light chain variable domains are displayed on the surface of filamentous phage. Combinations of variable domains are typically displayed on filamentous phage in the form of Fab's or scFvs. The library may be screened for phage bearing combinations of variable domains having desired antigen-binding characteristics. Preferred variable domain combinations are characterized by high affinity for a polypeptide encoded by the polynucleotides of NSEQ, or a portion thereof. Preferred variable domain combinations may also be characterized by high specificity for a polypeptide encoded by the polynucleotides of NSEQ, or a portion thereof, and little cross-reactivity to other related antigens. By screening from very large repertoires of antibody fragments, (2-10×10¹⁰) a good diversity of high affinity Mabs may be isolated, with many expected to have sub-nanomolar affinities for a polypeptide encoded by the polynucleotides of NSEQ, or a portion thereof.

Alternatively, human antibodies may be obtained from transgenic animals into which un-rearranged human Ig gene segments have been introduced and in which the endogenous mouse Ig genes have been inactivated. Preferred transgenic animals contain very large contiguous Ig gene fragments that are over 1 Mb in size but human polypeptide-specific Mabs of moderate affinity may be raised from transgenic animals containing smaller gene loci. Transgenic animals capable of expressing only human Ig genes may also be used to raise polyclonal antiserum comprising antibodies solely of human origin.

Antibodies of the invention may include those for which binding characteristics have been improved by direct mutation or by methods of affinity maturation. Affinity and specificity may be modified or improved by mutating CDRs and screening for antigen binding sites having the desired characteristics. CDRs may be mutated in a variety of ways. One way is to randomize individual residues or combinations of residues so that in a population of otherwise identical antigen binding sites, all twenty amino acids may be found at particular positions. Alternatively, mutations may be induced over a range of CDR residues by error prone PCR methods. Phage display vectors containing heavy and light chain variable region gene may be propagated in mutator strains of E. coli. These methods of mutagenesis are illustrative of the many methods known to one of skill in the art.

The antibody or antigen binding fragment may further comprise a detectable label (reporter molecule) attached thereto.

There is provided also methods of producing antibodies able to specifically bind to one of a polypeptide, polypeptide fragments, or polypeptide variants described herein, the method may comprise:
a) immunizing a mammal (e.g., mouse, a transgenic mammal capable of producing human Ig, etc.) with a suitable amount of a PSEQ described herein including, for example, a polypeptide fragment comprising at least 6 (e.g., 7, 8, 9, 10, 11, 12 etc. and up to the totality of the PSEQ) consecutive amino acids of a PSEQ;
b) collecting a fraction from blood, plasma or serum of the mammal, wherein the fraction comprises the antibody; and
c) isolating (e.g., substantially purifying) the polypeptide-specific antibodies from the serum of the mammal.

The method may further comprise the step of administering a second dose to the mammal (e.g., animal).

The antibody or antigen binding fragment (e.g., Fab) may thus be isolated in a purified or substantially purified form.

Methods of producing a hybridoma which secretes an antibody or antigen binding fragment that specifically binds to a polypeptide are also encompassed herewith and are known in the art.

The method may comprise:
a) immunizing a mammal (e.g., mouse, a transgenic mammal capable of producing human Ig, etc.) with a suitable amount of a PSEQ (including fragments) thereof;
b) obtaining lymphoid cells from the immunized animal obtained from (a);
c) fusing the lymphoid cells with an immortalizing cell to produce hybrid cells; and
d) selecting hybrid cells which produce antibody or antigen binding fragment that specifically binds to a PSEQ thereof.

Also encompassed by the present invention is a method of producing an antibody or antigen binding fragment that specifically binds to one of the polypeptide described herein, the method may comprise:
a) synthesizing a library of antibodies (e.g., antigen binding fragment) on phage or ribosomes or using commercial libraries;
b) panning the library against a sample by bringing the phage or ribosomes into contact with a composition comprising a polypeptide or polypeptide fragment described herein;
c) isolating phage which binds to the polypeptide or polypeptide fragment, and;
d) obtaining an antibody or antigen binding fragment from the phage or ribosomes.

The antibody or antigen binding fragment of the present invention may thus be obtained, for example, from a library (e.g., bacteriophage library) which may be prepared, for example, by
a) extracting cells which are responsible for production of antibodies from a host mammal;
b) isolating RNA from the cells of (a);
c) reverse transcribing mRNA to produce cDNA;
d) amplifying the cDNA using a (antibody-specific) primer; and
e) inserting the cDNA of (d) into a phage display vector or ribosome display cassette such that antibodies are expressed on the phage or ribosomes.

More particularly, the method may comprise a) contacting a library comprising a population of antibodies or antigen binding fragments with the polypeptide described herein, b) isolating an antibody or antigen binding fragment which is capable of specific binding with the polypeptide from the population and amplifying (amplification by PCR (e.g., RT-PCR), infection of bacteria with phages, etc.) a nucleic acid encoding the antibody or antigen binding fragment or a variable domain (light chain and/or heavy chain variable domain) of the antibody or antigen binding fragment.

The nucleic acid thus amplified may be used to transfect cells for producing an antibody or antigen binding fragment.

In order to generate antibodies, the host animal may be immunized with polypeptide and/or a polypeptide fragment and/or variant described herein to induce an immune response prior to extracting the cells which are responsible for production of antibodies.

The antibodies obtained by the means described herein may be useful for detecting proteins, variant and derivative polypeptides in specific tissues or in body fluids. Moreover, detection of aberrantly expressed proteins or protein fragments is probative of a disease state. For example, expression of the present polypeptides encoded by the polynucleotides of NSEQ, or a portion thereof, may indicate that the protein is being expressed at an inappropriate rate or at an inappropriate developmental stage. Hence, the present antibodies may be useful for detecting diseases associated with protein expression from NSEQs disclosed herein.

For in vivo detection purposes, antibodies may be those which preferably recognize an epitope present at the surface of a tumor cell.

A variety of protocols for measuring polypeptides, including ELISAs, RIAs, and FACS, are well known in the art and provide a basis for diagnosing altered or abnormal levels of expression. Standard values for polypeptide expression are established by combining samples taken from healthy subjects, preferably human, with antibody to the polypeptide under conditions for complex formation. The amount of complex formation may be quantified by various methods, such as photometric means. Quantities of polypeptide expressed in disease samples may be compared with standard values. Deviation between standard and subject values may establish the parameters for diagnosing or monitoring disease.

Design of immunoassays is subject to a great deal of variation and a variety of these are known in the art. Immunoassays may use a monoclonal or polyclonal antibody or antigen binding fragment that is directed against one epitope of the antigen being assayed. Alternatively, a combination of monoclonal or polyclonal antibodies may be used which are directed against more than one epitope. Protocols may be based, for example, upon competition where one may use competitive drug screening assays in which neutralizing antibodies capable of binding a polypeptide encoded by the polynucleotides of NSEQ, or a portion thereof, specifically compete with a test compound for binding the polypeptide. Alternatively one may use, direct antigen-antibody reactions or sandwich type assays and protocols may, for example, make use of solid supports or immunoprecipitation. Furthermore, antibodies may be labelled with a reporter molecule for easy detection. Assays that amplify the signal from a bound reagent are also known. Examples include immunoassays that utilize avidin and biotin, or which utilize enzyme-labelled antibody or antigen conjugates, such as ELISA assays.

Kits suitable for immunodiagnosis and containing the appropriate labelled reagents include antibodies directed against the polypeptide protein epitopes or antigenic regions, packaged appropriately with the remaining reagents and materials required for the conduct of the assay, as well as a suitable set of assay instructions.

The present invention therefore provides a kit for specifically detecting a polypeptide described herein, the kit may comprise, for example, an antibody or antibody fragment capable of binding specifically to the polypeptide described herein.

In accordance with the present invention, the kit may be a diagnostic kit, which may comprise:
a) one or more antibodies described herein; and
b) a detection reagent which may comprise a reporter group.

In accordance with the present invention, the antibodies may be immobilized on a solid support. The detection reagent may comprise, for example, an anti-immunoglobulin, protein G, protein A or lectin etc. The reporter group may be selected, without limitation, from the group consisting of radioisotopes, fluorescent groups, luminescent groups, enzymes, biotin and dye particles.

Additional aspects of the invention relates to isolated or substantially purified antibodies (including an antigen-binding fragment thereof) which may be capable of specifically binding to a polypeptide described herein.

The present invention thus provides an isolated or substantially purified antibody or an antigen binding fragment thereof which may comprise a light chain variable region and a heavy chain variable region capable of specific and non-covalent binding to the polypeptide described herein.

Exemplary and non-limitative embodiment of antibodies and antigen-binding fragments of the present invention are those which may bind to a polypeptide selected from the group consisting of;
a) A polypeptide which may comprise a sequence at least 75% identical to SEQ ID NO:3;
b) A polypeptide which may comprise a sequence at least 75% identical to SEQ ID NO:22;
c) A polypeptide which may comprise a sequence at least 75% identical to SEQ ID NO:23;
d) A polypeptide which may comprise a sequence at least 75% identical to SEQ ID NO:24,
e) A polypeptide which may comprise a fragment of from 6 to 143 consecutive amino acids of SEQ ID NO:3;
f) A polypeptide which may comprise a fragment of from 6 to 99 consecutive amino acids of SEQ ID NO:22;
g) A polypeptide which may comprise a fragment of from 6 to 74 consecutive amino acids of SEQ ID NO:23, and;
h) A polypeptide which may comprise a fragment of from 6 to 57 consecutive amino acids of SEQ ID NO:24.

More particularly, exemplary embodiments of the present invention relates to antibodies which may be capable of specifically binding a polypeptide comprising a polypeptide sequence encoded by SEQ ID NO:2 or a fragment of at least 6 amino acids of the polypeptide.

In yet an additional aspect, the present invention relates to a cell (e.g., hybridoma cell, CHO cells, etc.) which is capable of producing an antibody which may specifically bind to a polypeptide selected from the group consisting of;
a) a polypeptide which may comprise or consist in a polypeptide having a sequence at least 75% identical to SEQ ID NO:3, SEQ ID NO:22, SEQ ID NO:23 or SEQ ID NO:24 and;
b) a polypeptide which may comprise a polypeptide sequence encoded by any one of the polynucleotide sequence described herein or a fragment of at least 6 amino acids of the polypeptide.

The antibodies or antigen binding fragments which are particularly encompassed by the present invention are those for use in the detection of cancer cells (e.g., ovarian cancer cells, renal cancer cells or leukemia cells) or for use in the treatment, diagnosis or prognosis of cancer (e.g., ovarian cancer, renal cancer, leukemia).

Exemplary embodiments of cells which are more particularly encompassed by the present invention are those which may produce an antibody or an antigen binding fragment which may be capable of specifically binding a polypeptide comprising a polypeptide sequence encoded by SEQ ID NO:2 or a fragment of at least 6 amino acids of the polypeptide such as hybridoma. The antibodies or antigen binding fragments of the present invention may be those which are capable of specific binding to cancer cells (e.g., ovarian cancer cells, renal cancer cells, leukemia cells).

Other exemplary embodiments of the present invention relates to antibodies which are capable of specific binding to:
a) a polypeptide which may comprise or consist in a polypeptide having a sequence at least 75% identical to SEQ ID NO:3, SEQ ID NO:22, SEQ ID NO:23 or SEQ ID NO:24 and;
b) a polypeptide which may comprise a polypeptide sequence encoded by any one of the polynucleotide sequence described herein or a fragment of at least 6 amino acids of the polypeptide,
   provided that these antibodies do not bind to SEQ ID NO:32 and/or SEQ ID NO:33 with the same affinity or do not bind to SEQ ID NO:32 and/or 33.

As such regions of SEQ ID NO:3, 22, 23 or 24 which may be particularly of interest for the purpose of generating antibodies or antigen binding fragments are those which when compared (e.g., in an alignment) with SEQ ID NO:32 or SEQ ID NO:33 have at least one amino acid difference. Those selected region may comprise for example 6, 7, 8, 9, 10, 11, 12, 13, 14, 15 (or else) consecutive amino acids of SEQ ID NO:3 while the corresponding region of SEQ ID NO:32 and/or 33 will have at least one amino acid which is different or even all of the amino acid sequence will be different.

Not only, the amino acid sequence may be particularly selected for generating antibodies which preferentially binds to one protein target over another, but when the amino acid sequence selected (e.g., for screening, immunization, etc.) is common between two proteins (one being the preferential target and the other being a secondary target or an undesired target), one may select an antibody or antigen binding fragment from a population antibody or antigen binding fragment for its affinity toward the preferential target over the other secondary or an undesired target. Such selection may be performed in vitro using techniques known in the art. Such techniques may involve for example, seperately contacting the antibody or antigen binding fragment population with the preferential target amino acid sequence and the secondary or undesired target amino acid sequence and from the population, isolating the antibody or antigen binding fragment having a better affinity for the preferential target over the secondary or undesired target.

The present invention also relates to a composition which may comprise an antibody or an antigen binding fragment described herein. Encompassed by the present invention are pharmaceutical compositions comprising one or more of the antibodies or an antigen binding fragments described herein.

Other aspects of the invention relates to method of detecting cancer (cancer cells), treating cancer and/or diagnosing cancer using the antibody or antigen binding fragments described herein. The methods may comprise administering the antibody or antigen binding fragments described herein to a mammal in need (e.g., a mammal having or suspected of having cancer (e.g., ovarian cancer, renal cancer, leukemia). The antibody or antigen binding fragment of the present invention may be conjugated with a detectable moiety (i.e., for detection or diagnostic purposes) or with a therapeutic moiety (for therapeutic purposes)

A “detectable moiety” is a moiety detectable by spectroscopic, photochemical, biochemical, immunochemical, chemical and/or other physical means. A detectable moiety may be coupled either directly and/or indirectly (for example via a linkage, such as, without limitation, a DOTA or NHS linkage) to antibodies and antigen binding fragments thereof of the present invention using methods well known in the art. A wide variety of detectable moieties may be used, with the choice depending on the sensitivity required, ease of conjugation, stability requirements and available instrumentation. A suitable detectable moiety include, but is not limited to, a fluorescent label, a radioactive label (for example, without limitation, ¹²⁵I, In¹¹¹, Tc⁹⁹, I¹³¹ and including positron emitting isotopes for PET scanner etc), a nuclear magnetic resonance active label, a luminiscent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

In another exemplary embodiment of the invention, the antibody or antigen binding fragment thereof may be coupled (modified) with a therapeutic moiety (e.g., drug, cytotoxic moiety).

In an exemplary embodiment, the antibodies and antigen binding fragments may comprise a chemotherapeutic or cytotoxic agent. For example, the antibody and antigen binding fragments may be conjugated to the chemotherapeutic or cytotoxic agent. Such chemotherapeutic or cytotoxic agents include, but are not limited to, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷)). In other instances, the chemotherapeutic or cytotoxic agent may be comprised of, among others known to those skilled in the art, 5-fluorouracil, adriamycin, irinotecan, taxanes, pseudomonas endotoxin, ricin and other toxins.

Alternatively, in order to carry out the methods of the present invention and as known in the art, the antibody or antigen binding fragment of the present invention (conjugated or not) may be used in combination with a second molecule (e.g., a secondary antibody, etc.) which is able to specifically bind to the antibody or antigen binding fragment of the present invention and which may carry a desirable detectable, diagnostic or therapeutic moiety.

In a further aspect the present invention provides a method of making an antibody or an antigen binding fragment which may comprise immunizing a non-human animal with a polypeptide which may be selected, for example, from the group consisting of;
a) A polypeptide which may comprise a sequence at least 75% identical to SEQ ID NO:3;
b) A polypeptide which may comprise a sequence at least 75% identical to SEQ ID NO:22;
c) A polypeptide which may comprise a sequence at least 75% identical to SEQ ID NO:23;
d) A polypeptide which may comprise a sequence at least 75% identical to SEQ ID NO:24,
e) A polypeptide which may comprise a fragment of from 6 to 143 consecutive amino acids of SEQ ID NO:3;
f) A polypeptide which may comprise a fragment of from 6 to 99 consecutive amino acids of SEQ ID NO:22;
g) A polypeptide which may comprise a fragment of from 6 to 74 consecutive amino acids of SEQ ID NO:23, and;
h) A polypeptide which may comprise a fragment of from 6 to 57 consecutive amino acids of SEQ ID NO:24.

In accordance with the present invention, polypeptides which may comprise at least 75% identity with another polypeptide may generally have a length of at least 15 amino acids or more (i.e, at least 16, 17, 18 etc. including the total length of the protein or even larger polypeptides e.g., in fusions with BSA, KHL, etc.).

Detection of NSEQ and/or PSEQ

The present invention also relates to a method for identifying a cancer cell. The method may comprise contacting a cell, a cell sample (cell lysate), a body fluid (blood, urine, plasma, saliva etc.) or a tissue with a reagent which may be, for example, capable of specifically binding at least one NSEQ or PSEQ described herein. The method may more particularly comprise contacting a sequence isolated or derived from such cell, sample, fluid or tissue. The complex formed may be detected using methods known in the art.

In accordance with the present invention, the presence of the above mentioned complex may be indicative (a positive indication of the presence) of the presence of a cancer cell.

The present invention also relates in an additional aspect thereof to a method for the diagnosis or prognosis of cancer. The method may comprise, for example, detecting, in a cell, tissue, sample, body fluid, etc., at least one NSEQ or PSEQ described herein.

The cell, cell sample, body fluid or tissue may originate, for example, from an individual which has or is suspected of having a cancer and more particularly ovarian cancer, leukemia or renal cancer.

Any of the above mentioned methods may further comprise comparing the level obtained with at least one reference level or value.

Detection of NSEQ may require an amplification (e.g., PCR) step in order to have sufficient material for detection purposes. In accordance with the present invention, the polynucleotide described herein may comprise, for example, a RNA molecule, a DNA molecule, including those which are partial or complete, single-stranded or double-stranded, hybrids, modified by a group etc.

Other aspects of the present invention which are encompassed herewith comprises the use of at least one NSEQ or PSEQ described herein and derived antibodies in the manufacture of a composition for identification or detection of a cancer cell (e.g., a tumor cell) or for inhibiting or lowering the growth of cancer cell (e.g., for treatment of ovarian cancer or other cancer).

As some NSEQ and PSEQ are expressed at higher levels in malignant ovarian cancer than in LMP detection of such NSEQ or PSEQ in a sample from an individual (or in vivo) one may rule-out a low malignant potential ovarian cancer and may therefore conclude in a diagnostic of a malignant ovarian cancer. Furthermore, detection of the NSEQ or PSEQ in a cell, tissue, sample or body fluid from an individual may also be indicative of a late-stage malignant ovarian cancer. As such, therapies adapted for the treatment of a malignant ovarian cancer or a late-stage malignant ovarian cancer may be commenced.

In accordance with an embodiment of the present invention, the method may also comprise a step of qualitatively or quantitatively comparing the level (amount, presence) of at least one complex present in the test cell, test sample, test fluid or test tissue with the level of complex in a normal cell, a normal cell sample, a normal body fluid, a normal tissue or a reference value (e.g., for a non-cancerous condition).

The normal cell may be any cell which does not substantially express the desired sequence to be detected. Examples of such normal cells are included for example, in the description of the drawings section. A normal cell sample or tissue thus include, for example, a normal (non-cancerous) ovarian cell, a normal breast cell, a normal prostate cell, a normal lymphocyte, a normal skin cell, a normal renal cell, a normal colon cell, a normal lung cell and/or a normal cell of the central nervous system. For comparison purposes, a normal cell may be chosen from those of identical or similar cell type.

Of course, the presence of more than one complex may be performed in order to increase the precision of the diagnostic method. As such, at least two complexes (e.g., formed by a first reagent and a first polynucleotide and a second reagent or a second polynucleotide) or multiple complexes may be detected.

An exemplary embodiment of a reagent which may be used for detecting a NSEQ described herein is a polynucleotide which may comprise a sequence substantially complementary to the NSEQ.

A suitable reference level or value may be, for example, derived from the level of expression of a specified sequence in a low malignant potential ovarian cancer and/or from a normal cell.

It will be understood herein that a higher level of expression measured in a cancer cell, tissue or sample in comparison with a reference value or sample is indicative of the presence of cancer in the tested individual.

For example, the higher level measured in an ovarian cell, ovarian tissue or a sample of ovarian origin compared to a reference level or value for a normal cell (normal ovarian cell or normal non-ovarian cell) may be indicative of an ovarian cancer.

For comparison purpose, the presence or level of expression of a desired NSEQ or PSEQ to be detected or identified may be compared with the presence, level of expression, found in a normal cell which has been shown herein not to express the desired sequence.

In an additional aspect, the present invention relates to the use of at least one polypeptide in the manufacture of a composition for the identification or detection of a cancer cell (tumor cell). The polypeptide may be used, for example, as a standard in an assay and/or for detecting antibodies specific for the particular polypeptide, etc.

In yet an additional aspect, the present invention relates to the use of at least one polypeptide described herein in the identification or detection of a cancer cell, such as for example, an ovarian cancer cell or any other cancer cell as described herein.

The present invention therefore relates in a further aspect, to the use of at least one polypeptide described herein in the prognosis or diagnosis of cancer, such as, for example, a malignant ovarian cancer or a low malignant potential ovarian cancer.

As such and in accordance with the present invention, detection of the polypeptide in a cell (e.g., ovarian cell), tissue (e.g., ovarian tissue), sample or body fluid from an individual may preferentially be indicative of a malignant ovarian cancer diagnosis over a low malignant potential ovarian cancer diagnosis and therefore may preferentially be indicative of a malignant ovarian cancer rather than a low malignant potential ovarian cancer.

Further in accordance with the present invention, the presence of the polypeptide in a cell, tissue, sample or body fluid from an individual may preferentially be indicative of a late-stage malignant ovarian cancer.

There is also provided by the present invention, methods for identifying a cancer cell, which may comprise, for example, contacting a test cell, a test cell sample (cell lysate), a test body fluid (blood, urine, plasma, saliva etc.) or a test tissue with a reagent which may be capable of specifically binding the polypeptide or the nucleic acid described herein, and detecting the complex formed by the polypeptide or nucleic acid and reagent. The presence of a complex may be indicative (a positive indication of the presence) of a cancer cell such as for example, an ovarian cancer cell, leukemia, or a renal cancer cell.

The presence of a complex formed by the polypeptide or nucleic acid and the specific reagent may be indicative, for example, of ovarian cancer including, for example, a low malignant potential ovarian cancer or a malignant ovarian cancer.

However, the method is more particularly powerful for the detection of ovarian cancer of the malignant type. Therefore, the presence of a complex may preferentially be indicative of a malignant ovarian cancer relative (rather than) to a low malignant potential ovarian cancer.

Detection of the complex may also be indicative of a late stage malignant ovarian cancer.

In accordance with the present invention, the method may also comprise a step of qualitatively or quantitatively comparing the level (amount, presence) of at least one complex present in a test cell, a test sample, a test fluid or a test tissue with the level of complex in a normal cell, a normal cell sample, a normal body fluid, a normal tissue or a reference value (e.g., for a non-cancerous condition).

Of course, the presence of more than one polypeptide, nucleic acid or complex (two complexes or more (multiple complexes)) may be determined, e.g., one formed by a first specific reagent and a first polypeptide or nucleic acid and another formed by a second specific reagent and a second polypeptide or nucleic acid may be detected. Detection of more than one polypeptide or complex may help in the determination of the tumorigenicity of the cell.

An exemplary embodiment of a reagent, which may be used for the detection of the polypeptide described herein, is an antibody or antigen binding fragment.

An exemplary embodiment of a reagent, which may be used for the detection of the nucleic acid described herein, is a complement (e.g., a probe) of the nucleic acid sought to be detected.

The present invention also relates to a kit which may comprise at least one of the polypeptide described herein and/or a reagent capable of specifically binding to at least one of the polypeptide described herein.

### Use of NSEQ as a Screening Tool

The NSEQ described herein may be used either directly or in the development of tools for the detection and isolation of expression products (mRNA, mRNA precursor, hnRNA, etc.), of genomic DNA or of synthetic products (cDNA, PCR fragments, vectors comprising NSEQ etc.). Some NSEQs may also be used to prepare suitable tools for detecting an encoded polypeptide or protein. Some NSEQ may thus be used to provide an encoded polypeptide and to generate an antibody or an antigen binding fragment specific for the polypeptide.

Those skilled in the art will also recognize that short oligonucleotides sequences may be prepared based on the polynucleotide sequences described herein. For example, oligonucleotides having 10 to 20 nucleotides or more may be prepared for specifically hybridizing to a NSEQ having a substantially complementary sequence and to allow detection, identification and isolation of nucleic sequences by hybridization. Probe sequences of for example, at least 10-20 nucleotides may be prepared based on a sequence found in SEQ ID NO.:2 and more particularly selected from regions that lack homology to undesirable sequences. Probe sequences of 20 or more nucleotides that lack such homology may show an increased specificity toward the target sequence. Useful hybridization conditions for probes and primers are readily determinable by those of skill in the art. Stringent hybridization conditions encompassed herewith are those that may allow hybridization of nucleic acids that are greater than 90% identical but which may prevent hybridization of nucleic acids that are less than 70% identical. The specificity of a probe may be determined by whether it is made from a unique region, a regulatory region, or from a conserved motif. Both probe specificity and the stringency of diagnostic hybridization or amplification (maximal, high, intermediate, or low) reactions depend on whether or not the probe identifies exactly complementary sequences, allelic variants, or related sequences. Probes designed to detect related sequences may have, for example, at least 50% sequence identity to any of the selected polynucleotides.

Furthermore, a probe may be labelled by any procedure known in the art, for example by incorporation of nucleotides linked to a “reporter molecule”. A “reporter molecule”, as used herein, may be a molecule that provides an analytically identifiable signal allowing detection of a hybridized probe. Detection may be either qualitative or quantitative. Commonly used reporter molecules include fluorophores, enzymes, biotin, chemiluminescent molecules, bioluminescent molecules, digoxigenin, avidin, streptavidin or radioisotopes. Commonly used enzymes include horseradish peroxidase, alkaline phosphatase, glucose oxidase and β-galactosidase, among others. Enzymes may be conjugated to avidin or streptavidin for use with a biotinylated probe. Similarly, probes may be conjugated to avidin or streptavidin for use with a biotinylated enzyme. Incorporation of a reporter molecule into a DNA probe may be effected by any method known to the skilled artisan, for example by nick translation, primer extension, random oligo priming, by 3′ or 5′ end labeling or by other means. In addition, hybridization probes include the cloning of nucleic acid sequences into vectors for the production of mRNA probes. Such vectors are known in the art, are commercially available, and may be used to synthesize RNA probes in vitro. The labelled polynucleotide sequences may be used in Southern or northern analysis, dot blot, or other membrane-based technologies; in PCR technologies; and in micro arrays utilizing samples from subjects to detect altered expression. Oligonucleotides useful as probes for screening of samples by hybridization assays or as primers for amplification may be packaged into kits. Such kits may contain the probes or primers in a pre-measured or predetermined amount, as well as other suitably packaged reagents and materials needed for the particular hybridization or amplification protocol.

The expression of mRNAs identical or substantially identical to the NSEQs of the present invention may thus be detected and/or isolated using methods which are known in the art. Exemplary embodiment of such methods includes, for example and without limitation, hybridization analysis using oligonucleotide probes, reverse transcription and in vitro nucleic acid amplification methods.

Such procedures may therefore, permit detection of mRNAs in ovarian cells (e.g., ovarian cancer cells) or in any other cells expressing such mRNAs. Expression of mRNA in a tissue-specific or a disease-specific manner may be useful for defining the tissues and/or particular disease state. One of skill in the art may readily adapt the NSEQs for these purposes.

It is to be understood herein that the NSEQs may hybridize to a substantially complementary sequence found in a test sample (e.g., cell, tissue, etc.). Additionally, a sequence substantially complementary to NSEQ (including fragments) may bind a NSEQ and substantially identical sequences found in a test sample (e.g., cell, tissue, etc.).

Polypeptide encoded by an isolated NSEQ, polypeptide variants, or polypeptide fragments thereof are also encompassed herewith. The polypeptides whether in a premature, mature or fused form, may be isolated from lysed cells, or from the culture medium, and purified to the extent needed for the intended use. One of skill in the art may readily purify these proteins, polypeptides and peptides by any available procedure. For example, purification may be accomplished by salt fractionation, size exclusion chromatography, ion exchange chromatography, reverse phase chromatography, affinity chromatography and the like. Alternatively, PSEQ may be made by chemical synthesis.

Natural variants may be identified through hybridization screening of a nucleic acid library or polypeptide library from different tissue, cell type, population, species, etc. using the NSEQ and derived tools.

### Use of NSEQ for Development of an Expression System

In order to express a polypeptide, a NSEQ able to encode the PSEQ described herein may be inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, and in vivo genetic recombination.

A variety of expression vector/host cell systems known to those of skill in the art may be utilized to express a polypeptide or RNA from NSEQ. These include, but are not limited to, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems. For long-term production of recombinant proteins in mammalian systems, stable expression in cell lines may be effected. For example, NSEQ may be transformed into cell lines using expression vectors that may contain viral origins of replication and/or endogenous expression elements and a selectable or visible marker gene on the same or on a separate vector. The invention is not to be limited by the vector or host cell employed.

Alternatively, RNA and/or polypeptide may be expressed from a vector comprising NSEQ using an in vitro transcription system or a coupled in vitro transcription/translation system respectively.

In general, host cells that contain NSEQ and/or that express a polypeptide encoded by the NSEQ, or a portion thereof, may be identified by a variety of procedures known to those of skill in the art. These procedures include, but are not limited to, DNA/DNA or DNA/RNA hybridizations, PCR amplification, and protein bioassay or immunoassay techniques that include membrane, solution, or chip based technologies for the detection and/or quantification of nucleic acid or amino acid sequences. Immunological methods for detecting and measuring the expression of polypeptides using either specific polyclonal or monoclonal antibodies are known in the art. Examples of such techniques include enzyme-linked immunosorbent assays (ELISAs), radioimmunoassays (RIAs), and fluorescence activated cell sorting (FACS). Those of skill in the art may readily adapt these methodologies to the present invention.

Host cells comprising NSEQ may thus be cultured under conditions for the transcription of the corresponding RNA (mRNA, siRNA, shRNA etc.) and/or the expression of the polypeptide from cell culture. The polypeptide produced by a cell may be secreted or may be retained intracellularly depending on the sequence and/or the vector used. As will be understood by those of skill in the art, expression vectors containing NSEQ may be designed to contain signal sequences that direct secretion of the polypeptide through a prokaryotic or eukaryotic cell membrane. Due to the inherent degeneracy of the genetic code, other DNA sequences that encode the same, substantially the same or a functionally equivalent amino acid sequence may be produced and used, for example, to express a polypeptide encoded by NSEQ. The nucleotide sequences of the present invention may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In addition, a host cell strain may be chosen for its ability to modulate expression of the inserted sequences or to process the expressed polypeptide in the desired fashion. Such modifications of the polypeptide include, but are not limited to, acetylation, carboxylation, glycosylation, phosphorylation, lipidation, and acylation. Post-translational processing, which cleaves a “prepro” form of the polypeptide, may also be used to specify protein targeting, folding, and/or activity. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Those of skill in the art will readily appreciate that natural, modified, or recombinant nucleic acid sequences may be ligated to a heterologous sequence resulting in translation of a fusion polypeptide containing heterologous polypeptide moieties in any of the aforementioned host systems. Such heterologous polypeptide moieties may facilitate purification of fusion polypeptides using commercially available affinity matrices. Such moieties include, but are not limited to, glutathione S-transferase (GST), maltose binding protein, thioredoxin, calmodulin binding peptide, 6-His (His), FLAG, c-myc, hemaglutinin (HA), and antibody epitopes such as monoclonal antibody epitopes.

In yet a further aspect, the present invention relates to a polynucleotide which may comprise a nucleotide sequence encoding a fusion protein, the fusion protein may comprise a fusion partner fused to a peptide fragment of a protein encoded by, or a naturally occurring allelic variant polypeptide encoded by, the polynucleotide sequence described herein.

Those of skill in the art will also readily recognize that the nucleic acid and polypeptide sequences may be synthesized, in whole or in part, using chemical or enzymatic methods well known in the art. For example, peptide synthesis may be performed using various solid-phase techniques and machines such as the ABI 431A Peptide synthesizer (PE Biosystems) may be used to automate synthesis. If desired, the amino acid sequence may be altered during synthesis and/or combined with sequences from other proteins to produce a variant protein.

The present invention additionally relates to a bioassay for evaluating compounds as potential antagonists of the polypeptide described herein, the bioassay may comprise:
a) culturing test cells in culture medium containing increasing concentrations of at least one compound whose ability to inhibit the action of a polypeptide described herein is sought to be determined, wherein the test cells may contain a polynucleotide sequence described herein (for example, in a form having improved trans-activation transcription activity, relative to wild-type polynucleotide, and comprising a response element operatively linked to a reporter gene); and thereafter
b) monitoring in the cells the level of expression of the product of the reporter gene (encoding a reporter molecule) as a function of the concentration of the potential antagonist compound in the culture medium, thereby indicating the ability of the potential antagonist compound to inhibit activation of the polypeptide encoded by, the polynucleotide sequence described herein.

The present invention further relates to a bioassay for evaluating compounds as potential agonists for a polypeptide encoded by the polynucleotide sequence described herein, the bioassay may comprise:
a) culturing test cells in culture medium containing increasing concentrations of at least one compound whose ability to promote the action of the polypeptide encoded by the polynucleotide sequence described herein is sought to be determined, wherein the test cells may contain a polynucleotide sequence described herein (for example, in a form having improved trans-activation transcription activity, relative to wild-type polynucleotide, and comprising a response element operatively linked to a reporter gene); and thereafter
b) monitoring in the cells the level of expression of the product of the reporter gene as a function of the concentration of the potential agonist compound in the culture medium, thereby indicating the ability of the potential agonist compound to promote activation of a polypeptide encoded by the polynucleotide sequence described herein.

### Use of NSEQ as a Identification Tool or as a Diagnostic Screening Tool

The skilled artisan will readily recognize that NSEQ may be used to identify a particular cell, cell type, tissue, disease and thus may be used for diagnostic purposes to determine the absence, presence, or altered expression (i.e. increased or decreased compared to normal) of the expression product of a gene. Suitable NSEQ may be for example, between 10 and 20 or longer, i.e., at least 10 nucleotides long or at least 12 nucleotides long, or at least 15 nucleotides long up to any desired length and may comprise, for example, RNA, DNA, branched nucleic acids, and/or peptide nucleic acids (PNAs). In one alternative, the polynucleotides may be used to detect and quantify gene expression in samples in which expression of NSEQ is correlated with disease. In another alternative, NSEQ may be used to detect genetic polymorphisms associated with a disease. These polymorphisms may be detected, for example, in the transcript, cDNA or genomic DNA.

The invention provides for the use of the NSEQ described herein on an array and for the use of that array in a method of detection of a particular cell, cell type, tissue, disease for the prognosis or diagnosis of cancer. The method may comprise hybridizing the array with a patient sample (putatively comprising or comprising a target polynucleotide sequence substantially complementary to a NSEQ) under conditions to allow complex formation (between NSEQ and target polynucleotide), detecting complex formation, wherein the complex formation is indicative of the presence of the polynucleotide and wherein the absence of complex formation is indicative of the absence of the polynucleotide in the patient sample. The presence or absence of the polynucleotide may be indicative of cancer such as, for example, ovarian cancer or other cancer as indicated herein.

The method may also comprise the step of quantitatively or qualitatively comparing (e.g., with a computer system, apparatus) the level of complex formation in the patient sample to that of standards for normal cells or individual or other type, origin or grade of cancer.

The present invention provides one or more compartmentalized kits for detection of a polynucleotide and/or polypeptide for the diagnosis or prognosis of ovarian cancer. A first kit may have a receptacle containing at least one isolated NSEQ or probe comprising NSEQ. Such a probe may bind to a nucleic acid fragment which is present/absent in normal cells but which is absent/present in affected or diseased cells. Such a probe may be specific for a nucleic acid site that is normally active/inactive but which may be inactive/active in certain cell types. Similarly, such a probe may be specific for a nucleic acid site that may be abnormally expressed in certain cell types. Finally, such a probe may identify a specific mutation. The probe may be capable of hybridizing to the nucleic acid sequence which is mutated (not identical to the normal nucleic acid sequence), or may be capable of hybridizing to nucleic acid sequences adjacent to the mutated nucleic acid sequences. The probes provided in the present kits may have a covalently attached reporter molecule. Probes and reporter molecules may be readily prepared as described above by those of skill in the art.

### Use of NSEQ, PSEQ as a Therapeutic or Therapeutic Targets

One of skill in the art will readily appreciate that the NSEQ, PSEQ, expression systems, assays, kits and array discussed above may also be used to evaluate the efficacy of a particular therapeutic treatment regimen, in animal studies, in clinical trials, or to monitor the treatment of an individual subject. Once the presence of disease is established and a treatment protocol is initiated, hybridization or amplification assays may be repeated on a regular basis to determine if the level of mRNA or protein in the patient (patient's blood, tissue, cell etc.) begins to approximate the level observed in a healthy subject. The results obtained from successive assays may be used to show the efficacy of treatment over a period ranging from several days to many years.

In yet another aspect of the invention, NSEQ may be used therapeutically for the purpose of expressing mRNA and polypeptide, or conversely to block transcription and/or translation of the mRNA. Expression vectors may be constructed using elements from retroviruses, adenoviruses, herpes or vaccinia viruses, or bacterial plasmids, and the like. These vectors may be used for delivery of nucleotide sequences to a particular target organ, tissue, or cell population. Methods well known to those skilled in the art may be used to construct vectors to express nucleic acid sequences or their complements.

Alternatively, NSEQ may be used for somatic cell or stem cell gene therapy. Vectors may be introduced in vivo, in vitro, and ex vivo. For ex vivo therapy, vectors are introduced into stem cells taken from the subject, and the resulting transgenic cells are clonally propagated for autologous transplant back into that same subject. Delivery of NSEQ by transfection, liposome injections, or polycationic amino polymers may be achieved using methods that are well known in the art. Additionally, endogenous NSEQ expression may be inactivated using homologous recombination methods that insert an inactive gene sequence into the coding region or other targeted region of NSEQ.

Depending on the specific goal to be achieved, vectors containing NSEQ may be introduced into a cell or tissue to express a missing polypeptide or to replace a non-functional polypeptide. Of course, when one wishes to express PSEQ in a cell or tissue, one may use a NSEQ able to encode such PSEQ for that purpose or may directly administer PSEQ to that cell or tissue.

On the other hand, when one wishes to attenuate or inhibit the expression of PSEQ, one may use a NSEQ (e.g., an inhibitory NSEQ) which is substantially complementary to at least a portion of a NSEQ able to encode such PSEQ.

The expression of an inhibitory NSEQ may be done by cloning the inhibitory NSEQ into a vector and introducing the expression vector into a cell to down-regulate the expression of a polypeptide encoded by the target NSEQ. Complementary or anti-sense sequences may also comprise an oligonucleotide derived from the transcription initiation site; nucleotides between about positions −10 and +10 from the ATG may be used. Therefore, inhibitory NSEQ may encompass a portion which is substantially complementary to a desired nucleic acid molecule to be inhibited and a portion (sequence) which binds to an untranslated portion of the nucleic acid.

Similarly, inhibition may be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee et al. 1994)

Ribozymes, enzymatic RNA molecules, may also be used to catalyze the cleavage of mRNA and decrease the levels of particular mRNAs, such as those comprising the polynucleotide sequences of the invention. Ribozymes may cleave mRNA at specific cleavage sites. Alternatively, ribozymes may cleave mRNAs at locations dictated by flanking regions that form complementary base pairs with the target mRNA. The construction and production of ribozymes is well known in the art.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, may be included.

Pharmaceutical compositions are also encompassed by the present invention. The pharmaceutical composition may comprise at least one NSEQ or PSEQ and a pharmaceutically acceptable carrier.

As it will be appreciated form those of skill in the art, the specificity of expression NSEQ and/or PSEQ in tumor cells may advantageously be used for inducing an immune response (through their administration) in an individual having, or suspected of having a tumor expressing such sequence. Administration of NSEQ and/or PSEQ in individuals at risk of developing a tumor expressing such sequence is also encompassed herewith.

In addition to the active ingredients, a pharmaceutical composition may contain pharmaceutically acceptable carriers comprising excipients and auxiliaries that facilitate processing of the active compounds into preparations that may be used pharmaceutically.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and most preferably, humans.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

The term “treatment” for purposes of this disclosure refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

### Use of NSEQ in General Research

The invention also provides products, compositions, processes and methods that utilize a NSEQ described herein, a polypeptide encoded by a NSEQ described herein, a PSEQ described herein for research, biological, clinical and therapeutic purposes. For example, to identify splice variants, mutations, and polymorphisms and to generate diagnostic and prognostic tools.

NSEQ may be extended utilizing a partial nucleotide sequence and employing various PCR-based methods known in the art to detect upstream sequences such as promoters and other regulatory elements. Additionally, one may use an XL-PCR kit (PE Biosystems, Foster City Calif.), nested primers, and commercially available cDNA libraries (Life Technologies, Rockville Md.) or genomic libraries (Clontech, Palo Alto Calif.) to extend the sequence.

The polynucleotides (NSEQ) may also be used as targets in a microarray. The microarray may be used to monitor the expression patterns of large numbers of genes simultaneously and to identify splice variants, mutations, and polymorphisms. Information derived from analyses of the expression patterns may be used to determine gene function, to identify a particular cell, cell type or tissue, to understand the genetic basis of a disease, to diagnose a disease, and to develop and monitor the activities of therapeutic agents used to treat a disease. Microarrays may also be used to detect genetic diversity, single nucleotide polymorphisms which may characterize a particular population, at the genomic level.

The polynucleotides (NSEQ) may also be used to generate hybridization probes useful in mapping the naturally occurring genomic sequence. Fluorescent in situ hybridization (FISH) may be correlated with other physical chromosome mapping techniques and genetic map data.

It is to be understood herein that a sequence which is upregulated in an ovarian cancer cell (e.g., malignant ovarian cancer cell) may represent a sequence which is involved in or responsible for the growth, development, malignancy and so on, of the cancer cell (referred herein as a positive regulator of ovarian cancer). It is also to be understood that a sequence which is downregulated (unexpressed or expressed at low levels) in a malignant ovarian cancer cell may represent a sequence which is responsible for the maintenance of the normal status (untransformed) of an ovarian cell (referred herein as a negative regulator of ovarian cancer). Therefore, both the presence or absence of some sequences may be indicative of the disease or may be indicative of the disease, probability of having a disease, degree of severity of the disease (staging).

In a further aspect, the present invention relates to a method of identifying a compound which is capable of inhibiting the activity or function of a polypeptide which may be selected, for example from the group consisting of polypeptide having sequence at least 75% identical to SEQ ID NO:3 or a polypeptide comprising a polypeptide sequence encoded by SEQ ID NO:2. The method may comprise contacting the polypeptide with a putative compound an isolating or identifying a compound which is capable of specifically binding any one of the above mentioned polypeptide. The compound may originate from a combinatorial library.

The method may also further comprise determining whether the activity or function of the polypeptide is affected by the binding of the compound. Those compounds which capable of binding to the polypeptide and which and/or which are capable of altering the function or activity of the polypeptide represents a desirable compound to be used in cancer therapy.

The method may also further comprise a step of determining the effect of the putative compound on the growth of a cancer cell such as an ovarian cancer cell.

The present invention also relates to an assay and method for identifying a nucleic acid sequence and/or protein involved in the growth or development of ovarian cancer. The assay and method may comprise silencing an endogenous gene of a cancer cell such as an ovarian cancer cell and providing the cell with a candidate nucleic acid (or protein). A candidate gene (or protein) positively involved in inducing cancer cell death (e.g., apoptosis) (e.g., ovarian cancer cell) may be identified by its ability to complement the silenced endogenous gene. For example, a candidate nucleic acid involved in ovarian cancer modulation provided to a cell for which an endogenous gene has been silenced, may enable the cell to undergo apoptosis more so in the presence of an inducer of apoptosis.

Alternatively, an assay or method may comprise silencing an endogenous gene (gene expression) corresponding to the candidate nucleic acid or protein sequence to be evaluated and determining the effect of the candidate nucleic acid or protein on cancer growth (e.g., ovarian cancer cell growth). A sequence involved in the promotion or inhibition of cancer growth, development or malignancy may change the viability of the cell, may change the ability of the cell to grow or to form colonies, etc. The activity of a polypeptide may be impaired by targeting such polypeptide with an antibody or an antigen binding fragment or any other type of compound. Again, such compound may be identified by screening combinatorial libraries, phage libraries, etc.

The present invention also provides a method for identifying an inhibitory compound (inhibitor, antagonist) able to impair the function (activity) or expression of a polypeptide described herein. The method may comprise, for example, contacting the (substantially purified or isolated) polypeptide or a cell expressing the polypeptide with a candidate compound and measuring the function (activity) or expression of the polypeptide. A reduction in the function or activity of the polypeptide (compared to the absence of the candidate compound) may thus positively identify a suitable inhibitory compound.

The cell used to carry the screening test may be particularly chosen for the absence or low expression the polypeptide or variants described herein, or alternatively the expression of a naturally expressed polypeptide variant may be repressed.

It is to be understood herein, that if a “range” or “group” of substances (e.g. amino acids), substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example, with respect to a percentage (%) of identity of from about 80 to 100%, it is to be understood as specifically incorporating herein each and every individual %, as well as sub-range, such as for example 80%, 81%, 84.78%, 93%, 99% etc. with respect to a length of “about 10 to about 25” it is to be understood as specifically incorporating each and every individual number such as for example 10, 11, 12, 13, 14, 15 up to and including 25; and similarly with respect to other parameters such as, concentrations, elements, etc.

Other objects, features, advantages, and aspects of the present invention will become apparent to those skilled in the art from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### Exemplary Embodiments

The applicant employed a carefully planned strategy to identify and isolate genetic sequences involved in cancer control, growth, development or else.

Key to the discovery of differentially expressed sequences unique to malignant ovarian cancer is the use of the applicant's patented STAR technology (Subtractive Transcription-based Amplification of mRNA; U.S. Pat. No. 5,712,127 Malek et al., 1998). Based on this procedure, mRNA isolated from malignant ovarian tumor sample is used to prepare “tester RNA”, which is hybridized to complementary single-stranded “driver DNA” prepared from mRNA from LMP sample and only the un-hybridized “tester RNA” is recovered, and used to create cloned cDNA libraries, termed “subtracted libraries”. Thus, the “subtracted libraries” are enriched for differentially expressed sequences inclusive of rare and novel mRNAs often missed by micro-array hybridization analysis. These rare and novel mRNA are thought to be representative of important gene targets for the development of better diagnostic and therapeutic strategies.

The clones contained in the enriched “subtracted libraries” are identified by DNA sequence analysis and their potential function assessed by acquiring information available in public databases (NCBI and GeneCard). The non-redundant clones are then used to prepare DNA micro-arrays, which are used to quantify their relative differential expression patterns by hybridization to fluorescent cDNA probes. Two classes of cDNA probes may be used, those which are generated from either RNA transcripts prepared from the same subtracted libraries (subtracted probes) or from mRNA isolated from different ovarian LMP and malignant samples (standard probes). The use of subtracted probes provides increased sensitivity for detecting the low abundance mRNA sequences that are preserved and enriched by STAR. Furthermore, the specificity of the differentially expressed sequences to malignant ovarian cancer is measured by hybridizing radio-labeled probes prepared from each selected sequence to macroarrays containing RNA from different LMP and malignant ovarian cancer samples and different normal human tissues.

A major challenge in gene expression profiling is the limited quantities of RNA available for molecular analysis. The amount of RNA isolated from many human specimens (needle aspiration, laser capture micro-dissection (LCM) samples and transfected cultured cells) is often insufficient for preparing: 1) conventional tester and driver materials for STAR; 2) standard cDNA probes for DNA micro-array analysis; 3) RNA macroarrays for testing the specificity of expression; 4) Northern blots and; 5) full-length cDNA clones for further biological validation and characterization etc. Thus, the applicant has developed a proprietary technology called RAMP (RNA Amplification Procedure) (U.S. patent application Ser. No. 11/000,958 published under No. US 2005/0153333A1 on Jul. 14, 2005 and entitled “Selective Terminal Tagging of Nucleic Acids”), which linearly amplifies the mRNA contained in total RNA samples yielding microgram quantities of amplified RNA sufficient for the various analytical applications. The RAMP RNA produced is largely full-length mRNA-like sequences as a result of the proprietary method for adding a terminal sequence tag to the 3′-ends of single-stranded cDNA molecules, for use in linear transcription amplification. Greater than 99.5% of the sequences amplified in RAMP reactions show <2-fold variability and thus, RAMP provides unbiased RNA samples in quantities sufficient to enable the discovery of the unique mRNA sequences involved in ovarian cancer.

The process for identifying sequences involved in ovarian cancer with such great reliability involved the following steps which are outlined in details in international application No: PCT/CA2007/001134: 1) preparation of highly representative cDNA libraries using mRNA isolated from LMPs and malignant ovarian cancer samples of human origin; 2) isolation of sequences upregulated in the malignant ovarian cancer samples; 3) identification and characterization of upregulated sequences; 4) selection of upregulated sequences for tissue specificity; 5) determination of knock-down effects on ovarian cancer cell line proliferation and migration; and 6) determination of the expression pattern of each upregulated sequence in samples derived from nine different cancer types. The results obtained so far using this technology demonstrate the advantage of targeting ovarian cancer-related genes that are highly specific to this differentiated cell type compared to normal tissues and provide a more efficient screening method when studying the genetic basis of diseases and disorders.

TABLE 3
shows the pathologies including grade and stage of the different
ovarian cancer samples used on the macroarrays for testing the
differentially expressed sequences.

| MF | | | | | Position |
|---|---|---|---|---|---|
| Code | | | | | on Macro- |
| No. | Pathologies | Symbol | Stage | Grade | array |
| 15 | Borderline serous | B | 1b | B | A1 |
| 16 | Borderline serous | B | 2a | B | B1 |
| 17 | Borderline/carcinoma | B/CS | 3c | 1 | F1 |
| | serous | | | | |
| 18 | Borderline serous | B | 3c | B | C1 |
| 19 | Borderline serous | B | 1b | B | D1 |
| 20 | Borderline serous | B | 1a | B | E1 |
| 42 | Carcinoma serous of | CSS | 3a | 3 | A4 |
| | the surface | | | | |
| 22 | Carcinoma serous | CS | 1b | 3 | A2 |
| 30 | Carcinoma serous | CS | 2c | 3 | E2 |
| 23 | Carcinoma serous | CS | 3c | 3 | F2 |
| 25 | Carcinoma serous | CS | 3c | 3 | B2 |
| 26 | Carcinoma serous | CS | 3c | 3 | A3 |
| 27 | Carcinoma serous | CS | 3c | 3 | C2 |
| 28 | Carcinoma serous | CS | 3c | 3 | D2 |
| 43 | Carcinoma serous | CS | 3c | 3 | B4 |
| 45 | Carcinoma serous | CS | 3c | 3 | D4 |
| 49 | Carcinoma serous | CS | 3c | 2 | F4 |
| 41 | Carcinoma endometrioide | CE | 3b | 3 | G3 |
| 40 | Carcinoma endometrioide | CE | 3c | 3 | F3 |
| 44 | Carcinoma endometrioide | CE | 3c | 3 | C4 |
| 39 | Carcinoma endometrioide | CE | 3c | 2 | E3 |
| 50 | Carcinoma endometrioide | CE | 1c | 1 | G4 |
| 46 | Carcinoma endometrioide | CE | 1a | 2 | E4 |
| 34 | Clear cell carcinoma | CCC | 3c | 2 | B3 |
| 38 | Clear cell carcinoma | CCC | 3c | 3 | D3 |
| 37 | Clear cell carcinoma | CCC | 1c | 2 | C3 |

A sequence of particular interest and described in international application No. PCT/CA2007/001134 (herein referred as SEQ ID NO:1) was obtained using this methodology. We have investigated further on that sequence.

### Determining Malignant Ovarian Cancer Specificity of the Differentially Expressed Sequences Identified:

The differentially expressed sequences (SEQ ID NO:1 and/or SEQ ID NO:2 or any fragments and complements thereof) are tested for specificity by hybridization to nylon membrane-based macroarrays. The macroarrays are prepared using RAMP amplified RNA from 6 LMP and 20 malignant human ovarian samples, and 30 normal human tissues (adrenal, liver, lung, ovary, skeletal muscle, heart, cervix, thyroid, breast, placenta, adrenal cortex, kidney, vena cava, fallopian tube, pancreas, testicle, jejunum, aorta, esophagus, prostate, stomach, spleen, ileum, trachea, brain, colon, thymus, small intestine, bladder and duodenum) purchased commercially (Ambion, Austin, Tex.). In addition, RAMP RNA prepared from breast cancer cell lines, MDA and MCF7, prostate cancer cell line, LNCap, and a normal and prostate cancer LCM microdissected sample. In case of limited quantities of mRNA available for, it may be necessary to first amplify the mRNA using the RAMP methodology. Each amplified RNA sample are reconstituted to a final concentration of 250 ng/μL in 3×SSC and 0.1% sarkosyl in a 96-well microtitre plate and 1 μL spotted onto Hybond N+ nylon membranes using the specialized MULTI-PRINT™ apparatus (VP Scientific, San Diego, Calif.), air dried and UV-cross linked. The sequences selected are radiolabeled with α-³²P-dCTP using the random priming procedure recommended by the supplier (Amersham, Piscataway, N.J.) and used as probes on the macroarrays. Hybridization and washing steps are performed following standard procedures well known to those skilled in the art.

Using the same RAMP RNA samples that are spotted on the macroarrays, 500 μg of RNA are converted to single-stranded cDNA with Thermoscript RT (Invitrogen, Burlington, ON) as described by the manufacturer. The cDNA reaction are diluted so that 1/200 of the reaction is used for each PCR experiment. After trial PCR reactions with gene-specific primers designed against each SEQ. ID NOs. to be tested, the linear range of the reaction is determined and applied to all samples, PCR is conducted in 96-well plates using Hot-Start Taq Polymerase from Qiagen (Mississauga, ON) in a DNA Engine Tetrad from MJ Research. Half of the reaction mixture is loaded on a 1.2% agarose/ethidium bromide gel and the amplicons visualized with UV light.

Alternatively, complementary DNAs is prepared using, for example, random hexamers from RAMP amplified RNA from human LMP samples and malignant ovarian tumor samples (Table 3). The cDNAs are quantified and used as templates for PCR with gene-specific primers using standard methods known to those skilled in the art.

A primer pair, OGS 1212 (AAGCATAGCCATAGGTGATTGG; SEQ. ID. NO. 20) and OGS 1213 (ACAGGTATCAGACAAGGGAGCAG; SEQ. ID. NO. 21) for SEQ. ID. NO. 1 may be used to perform RT-PCR on LMP samples, different stages/grades of ovarian cancer and normal human tissue samples.

### SEQ. ID. NO:1

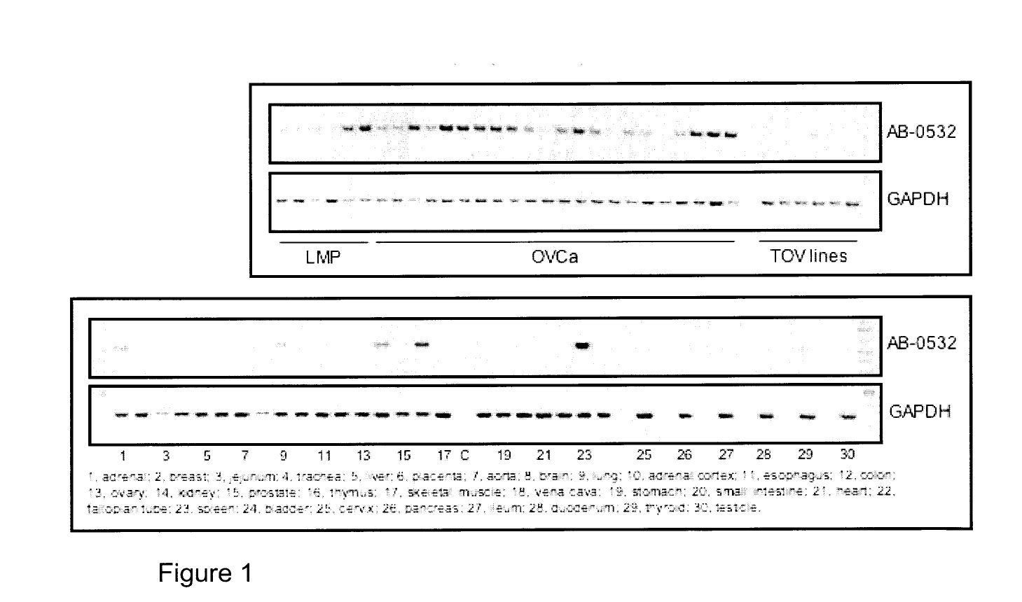

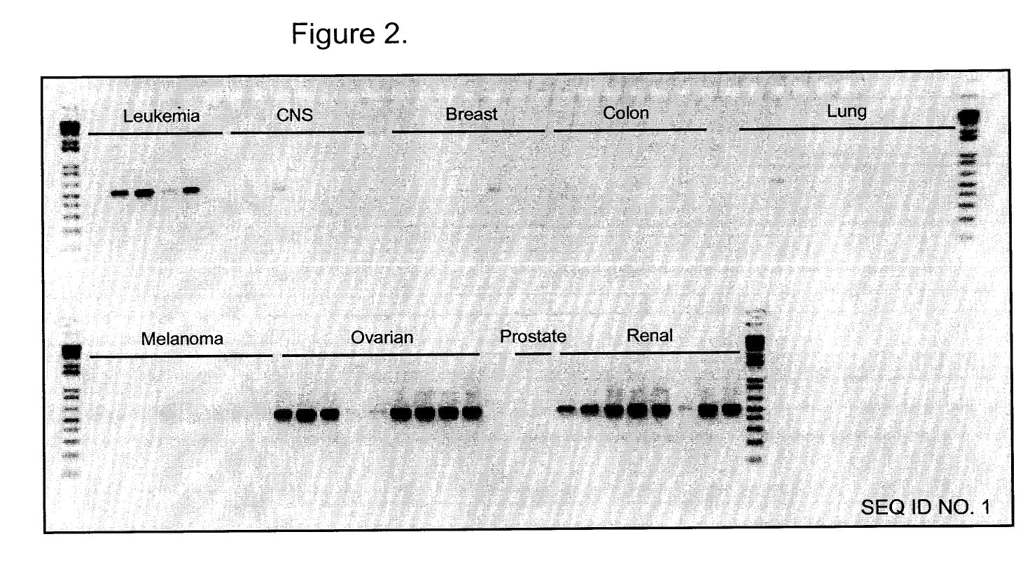

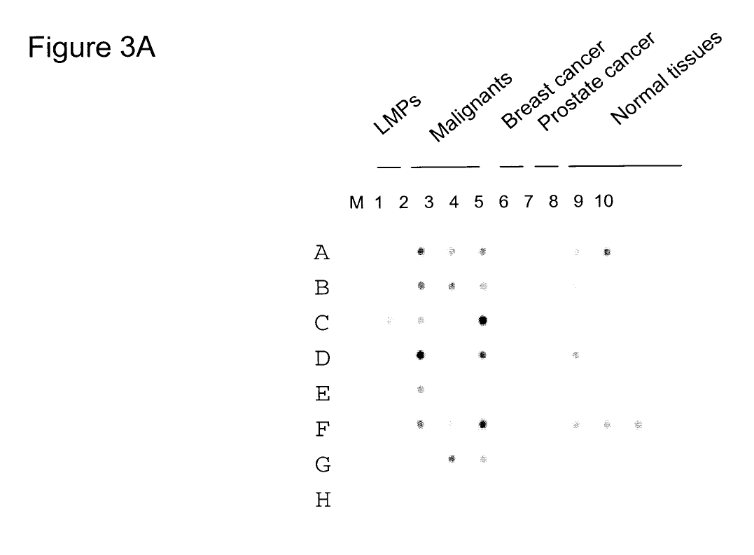

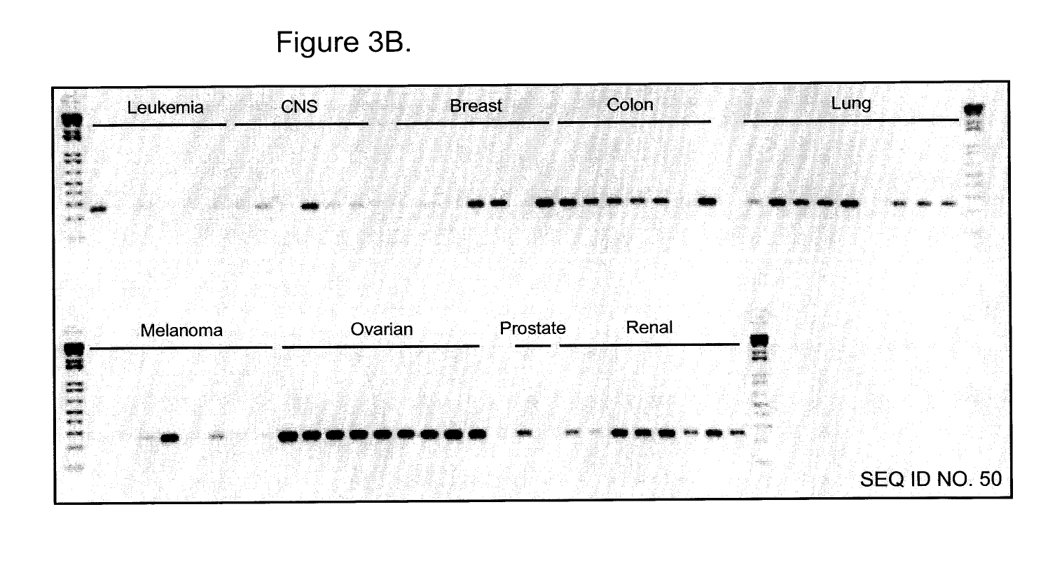

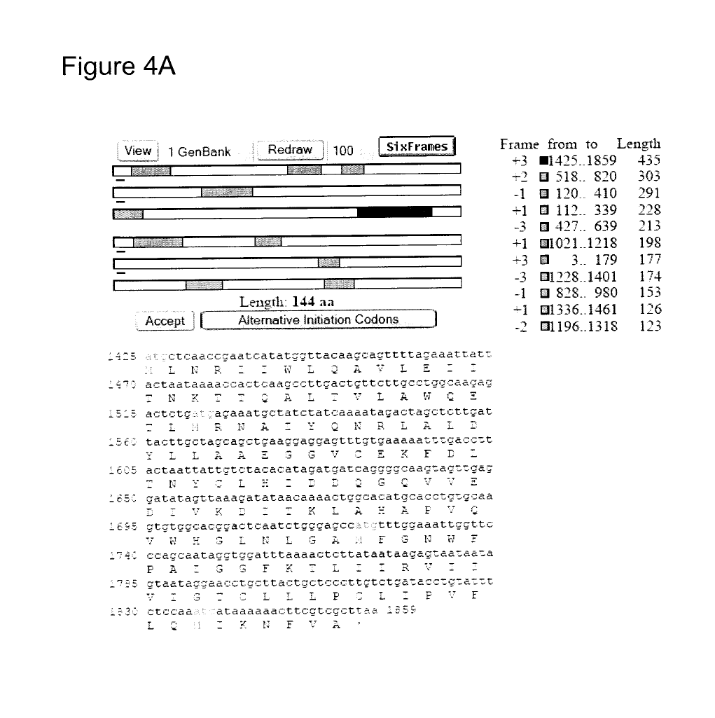

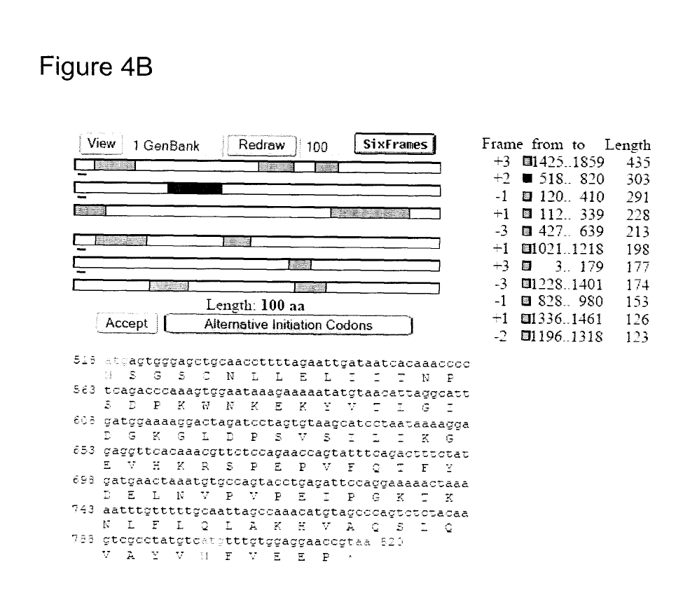

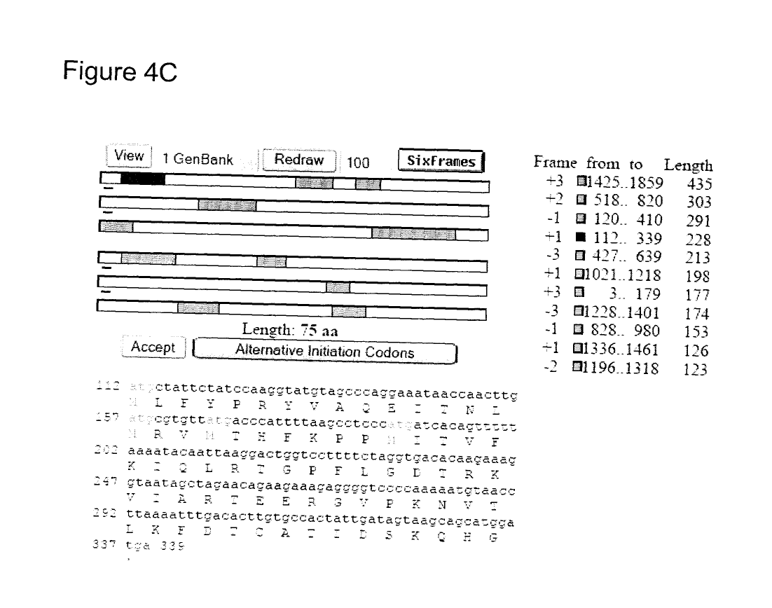

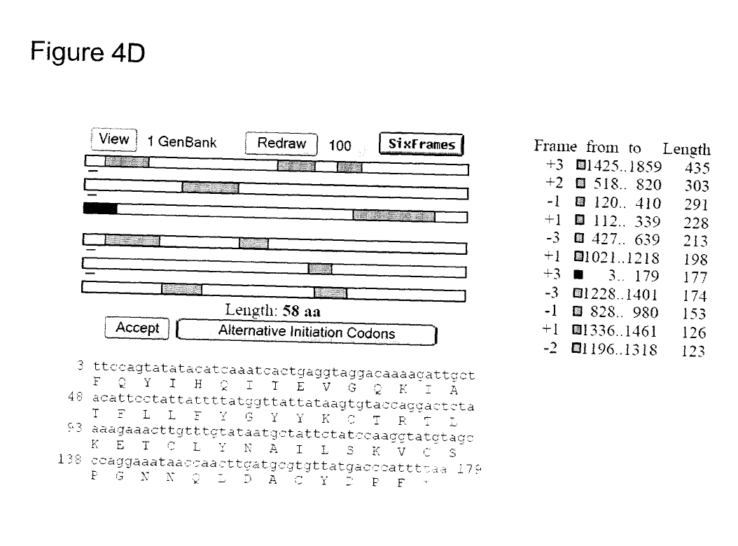

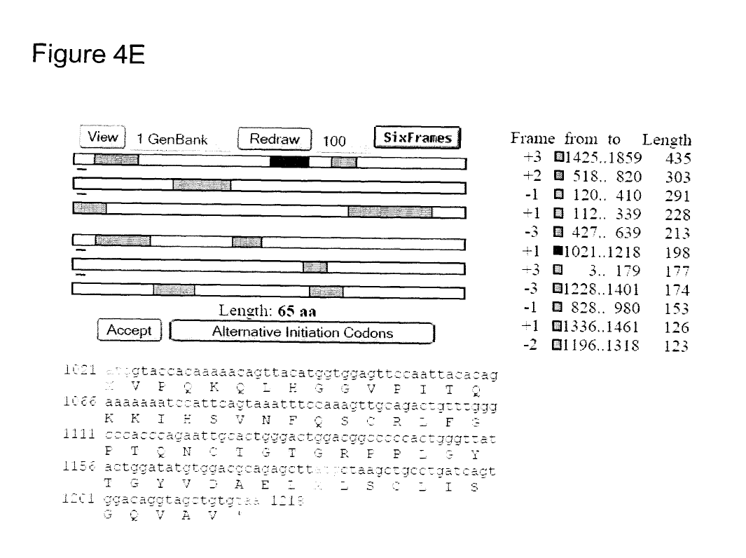

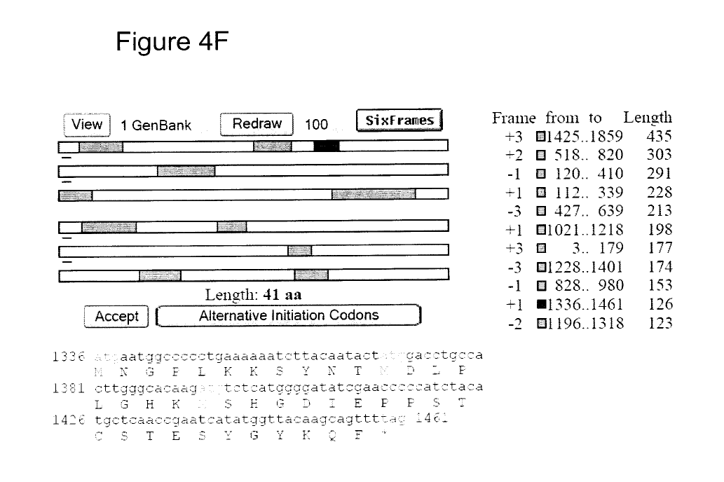

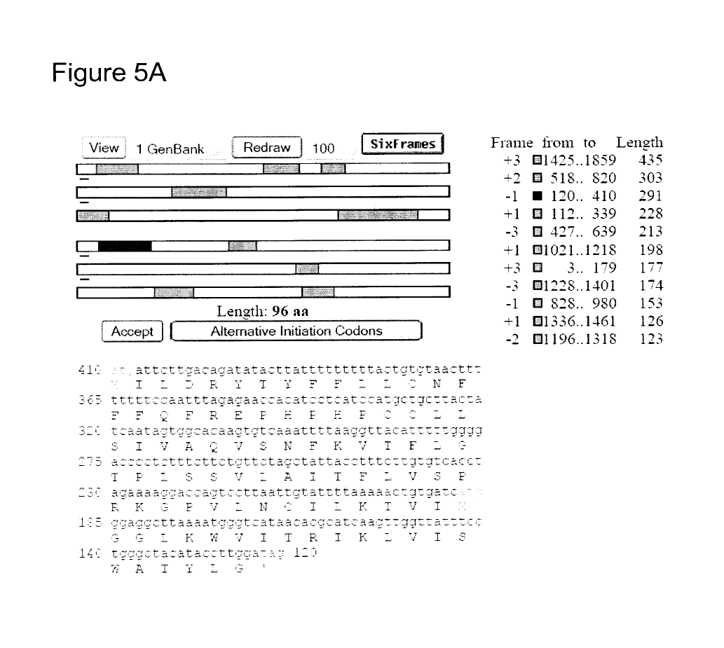

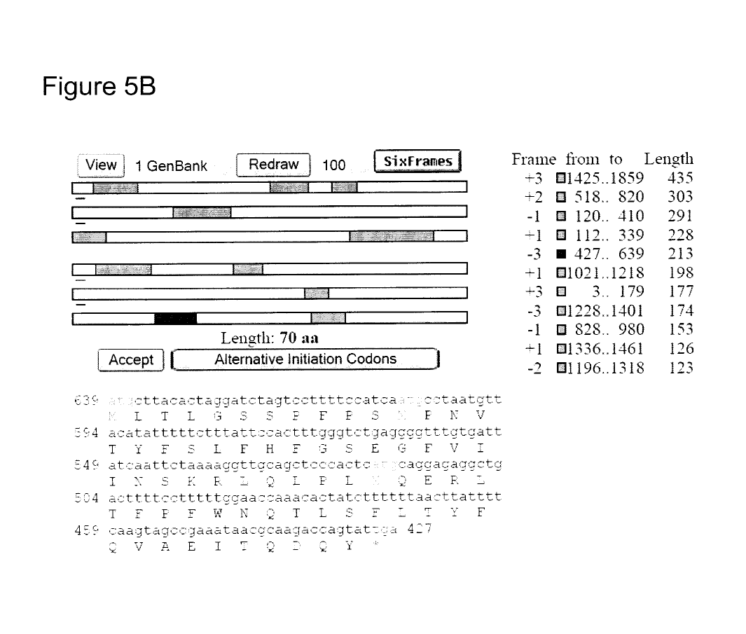

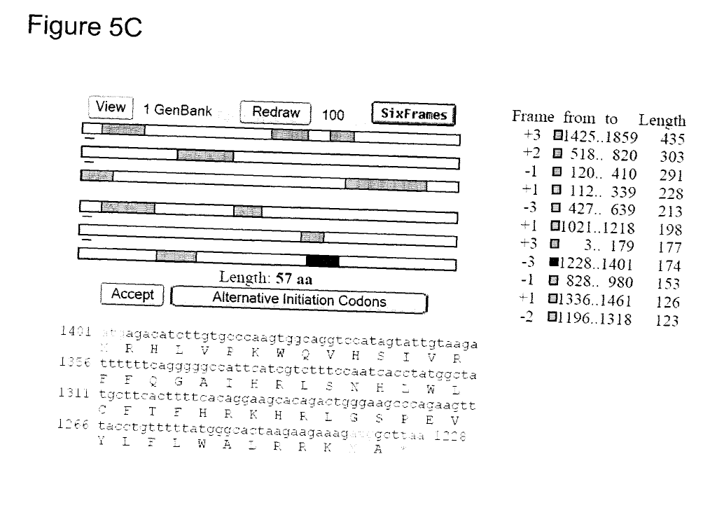

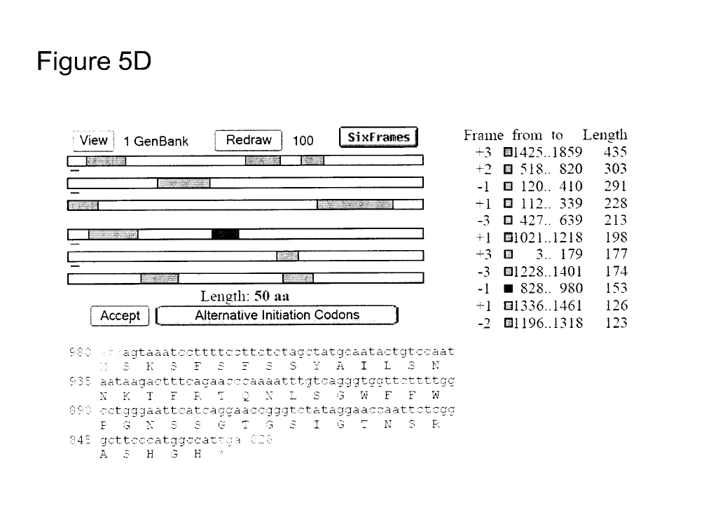

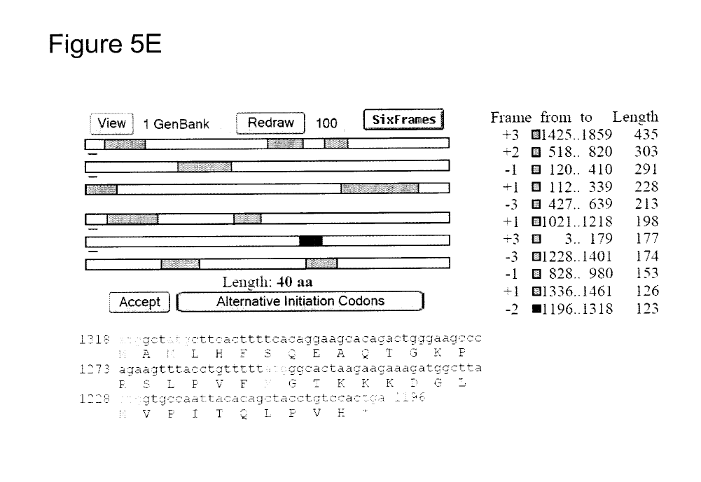

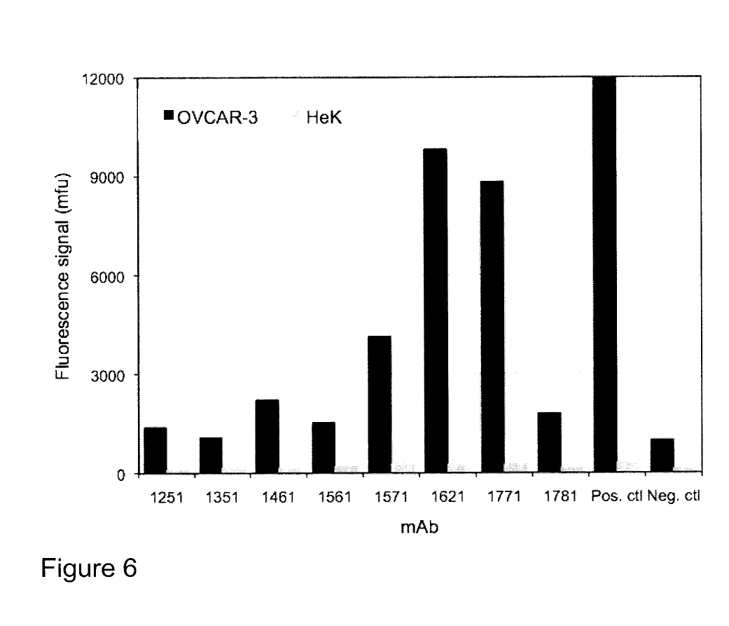

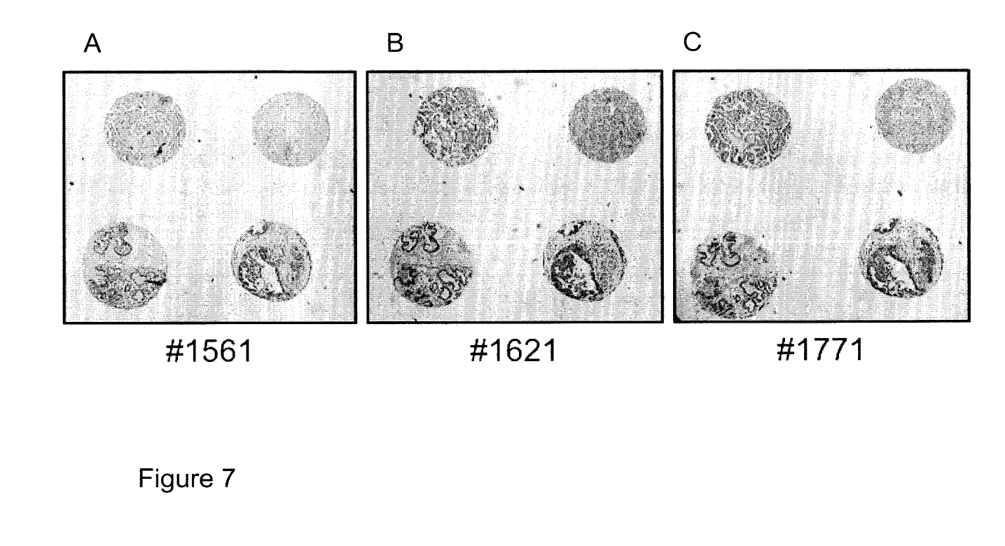

The STAR sequence represented by the isolated SEQ. ID. NO:1 maps to chromosome 1, and may represent a portion of an unknown gene sequence (see Table 2). Weak homology has been found between SEQ. ID. NO. 1 and the envelop proteins present at the surface of human endogenous retroviruses. We have demonstrated that this STAR clone sequence is markedly upregulated in malignant ovarian cancer samples compared to ovarian LMP samples and a majority of normal human tissues (FIG. 1), which have not been previously reported. Thus, it is believed that expression of the gene corresponding to this STAR sequence (and polynucleotides comprising this STAR sequence) may be required for, or involved in ovarian cancer tumorigenesis.

RT-PCR results obtained for SEQ ID NO: 1 (identified as SEQ ID NO:41 in PCT/CA2007/001134) indicated that this sequence is specifically expressed in cancer cells (of ovarian cancer, renal cancer and leukemia). This has prompted us to investigate further on the sequence.

### SEQ ID NO:2

A longer transcript containing SEQ ID NO:1 was isolated using standard technology (SEQ ID NO:2). Upon performing an homology search with fragment 1425 to 1859 of SEQ ID NO:2 using the Blast program (NCBI), we have identified two transcripts sharing 91% identity with fragment 1425 to 1853 or 1425 to 1859 of SEQ ID NO:2 (NCBI accession Nos. XM_{—}942991.3 and XM_{—}001723876.1 respectively). When using the complete SEQ ID NO:2 sequence, we found that both of these transcripts share 91% identity (or 754 common nucleotides) over a fragment covering nucleotide 1200 to 2021 of SEQ ID NO:2, i.e., 37% identity over the entire length of SEQ ID NO:2.

Upon searching for open reading frames we identified 4 ORFs starting in different frames (+1, +2 or +3), namely SEQ ID NO:3 (+3 frame), SEQ ID NO: 22 (+2 frame), SEQ ID NO:23 (+1 frame) and SEQ ID NO:24 (+3 frame). Each of these sequence encodes fragments having significant homology with the env gene of a human endogenous retrovirus known as ERV3 or HERV-R (see Table 4).

TABLE 4

| Polypeptide | % identity with env | % similarity with env | |
|---|---|---|---|
| (length) | gene of ERV3 | gene of ERV3 | Closest analogue |
| SEQ ID NO: 3 | 73% | 86% | Acc. No. AAP06678.1 |
| (144 amino acids) | 106 amino acids over | 124 amino acids over | Acc. No. AAP29640.1 |
| | 144 are identical to | 144 are identical or | (SEQ ID NO: 32) |
| | closest analogue | similar to closest | |
| | | analogue | |
| | 72% | 86% | Acc. No. EAW74675.1 |
| | 105 amino acids out of | 124 amino acids over | (SEQ ID NO: 33) |
| | 144 are identical to | 144 are identical or | |
| | closest analogue | similar to closest | |
| | | analogue | |
| SEQ ID NO: 22 | 71% | 84% | Acc. No. AAP06678.1 |
| (100 amino acids) | 64 amino acids over 90 | 76 amino acids over 90 | Acc. No. AAP29640.1 |
| | are identical to closest | are identical or similar | |
| | analogue | to closest analogue | |
| | 64% over the entire | 76% over the entire | |
| | length of SEQ ID NO: 3. | length of SEQ ID NO: 3. | |
| SEQ ID NO: 23 | 58% | 77% | Acc. No. AAP06678.1 |
| (75 amino acids) | 31 amino acids over 53 | 41 amino acids over 53 | Acc. No. AAP29640.1 |
| | are identical to closest | are identical or similar | |
| | analogue | to closest analogue | |
| | 41% over the entire | 55% over the entire | |
| | length of SEQ ID NO: 3. | length of SEQ ID NO: 3. | |
| SEQ ID NO: 24 | 67% | 78% | Acc. No. AAP06678.1 |
| (58 amino acids) | 38 amino acids over 56 | 44 amino acids over 56 | Acc. No. AAP29640.1 |
| | are identical to closest | are identical or similar | |
| | analogue | to closest analogue | |
| | 66% over the entire | 76% over the entire | |
| | length of SEQ ID NO: 3. | length of SEQ ID NO: 3. | |

Several antibodies were generated or selected against SEQ ID NO:3 and a few of them were tested for their specificity and affinity against ovarian cancer cells in comparison with normal ovarian cells.

Cytometry (i.e., fluoresence-activated cell sorting (FACS)) was performed on OVCAR-3 ovarian cancer cell lines using the antibodies (Fabs) generated against SEQ ID NO:3.

Briefly, OVCAR-3 ovarian cancer cells were catured by antibodies specific for SEQ ID NO:3. Results illustrated in FIG. 6 indicate that the strongest binders were the #1621 and #1771 mAbs whereas the #1561 was among the weakest. The negative control cell line was the HEK-293 human cell line which does not express SEQ ID NO:3.

Immunohistochemistry was also conducted using 3 antibodies as follows. Paraffin-embedded epithelial ovarian tumor samples (all serous histotypes) were placed on glass slides and fixed for 15 min at 50° C. Deparaffinization was conducted by treating 2× with xylene followed by dehydration in successive 5 min washes in 100%, 80%, and 70% ethanol. The slides were washed 2× in PBS for 5 min and treated with antigen retrieval solution (citrate-EDTA) to unmask the antigen. Endogenous peroxide reactive species were removed by incubating slides with H₂O₂ in methanol and blocking was performed by incubating the slides with serum-free blocking solution (Dakocytomation) for 20 min at room temperature. The antibodies (#1561, #1621, and #1771 respectively) were added for 1 h at room temperature. Antibody-reactive antigen was detected by incubating with biotin-conjugated mouse anti-kappa followed by streptavidin-HRP tertiary antibody. Positive staining was revealed by treating the slides with DAB-hydrogen peroxide substrate for less than 5 min and subsequently counterstained with hematoxylin.

The results of FIG. 7 showed that positive staining was observed in the epithelial layer of the ovarian tumors. The stromal layer was negative. Additionally, the intensity of staining was strongest with the #1621 and #1771 antibodies compared to the #1561 antibodies. This was in agreement with cell sorting studies that showed the same correlation in the ability of the antibodies to sort OVCAR-3 ovarian cancer cells.

The results illustrated in FIG. 6 and FIG. 7 thus indicate that SEQ ID NO:3 is specifically expressed in ovarian cancer cells and may therefore be used as targets for the treatment, detection and/or diagnosis of ovarian cancer.

These experiments therefore provides a first demonstrated evidence that the open reading frames encoded by SEQ ID NO:2 are translated into protein sequences of which expression is associated with cancer.

It is possible that the other ERV3-related open reading frames or even non-coding sequences may also be involved in the development and/or progression of cancer.

### SEQ. ID. NO:4

The candidate protein encoded by the isolated SEQ. ID. NO:4 is a previously identified gene that encodes a protein, Folate receptor 1 (adult) (FOLR1), with members of this gene family having a high affinity for folic acid and for several reduced folic acid derivatives, and mediate delivery of 5-methyltetrahydrofolate to the interior of cells (see Table 6). We have demonstrated that this gene is markedly upregulated in malignant ovarian cancer samples compared to ovarian LMP samples and a majority of normal human tissues (FIG. 3A). The potential role of FOLR1 in ovarian cancer therapeutics has been previously documented (Leamon and Low, 2001 and Jhaveri et al., 2006, U.S. Pat. No. 7,030,236). By way of example of the FOLR1 gene target, similar genes described herein with upregulation in malignant ovarian tumors and limited or no expression in a majority of normal tissues may also serve as potential therapeutic targets for ovarian cancer.

### RNA Interference Studies

RNA interference is a recently discovered gene regulation mechanism that involves the sequence-specific decrease in a gene's expression by targeting the mRNA for degradation and although originally described in plants, it has been discovered across many animal kingdoms from protozoans and invertebrates to higher eukaryotes (reviewed in Agrawal et al., 2003). In physiological settings, the mechanism of RNA interference is triggered by the presence of double-stranded RNA molecules that are cleaved by an RNAse III-like protein active in cells, called Dicer, which releases the 21-23 bp siRNAs. The siRNA, in a homology-driven manner, complexes into a RNA-protein amalgamation termed RISC(RNA-induced silencing complex) in the presence of mRNA to cause degradation resulting in attenuation of that mRNA's expression (Agrawal et al., 2003).

Current approaches to studying the function of genes, such as gene knockout mice and dominant negatives, are often inefficient, and generally expensive, and time-consuming. RNA interference is proving to be a method of choice for the analysis of a large number of genes in a quick and relatively inexpensive manner. Although transfection of synthetic siRNAs is an efficient method, the effects are often transient at best (Hannon G. J., 2002). Delivery of plasmids expressing short hairpin RNAs by stable transfection has been successful in allowing for the analysis of RNA interference in longer-term studies (Brummelkamp et al., 2002; Elbashir et al., 2001).

### Determination of Knockdown Effects on the Proliferation of Ovarian Cancer Cell Lines

In order to determine which ovarian cancer-specific genes participate in the proliferation of ovarian cancer cells, an assay was developed using stably transfected cell lines that contain attenuated (i.e., knocked down) levels of the specific gene being investigated. Two human ovarian cancer cell lines derived from chemotherapy-naïve patients were utilized that have been previously characterized in terms of their morphology, tumorigenicity, and global expression profiles. In addition, these analyses revealed that these cell lines were excellent models for in vivo behavior of ovarian tumors in humans (Provencher et al., 2000 and Samouelian et al., 2004). These cell lines are designated TOV-21G and TOV-112D.

The design and subcloning of individual shRNA expression cassettes and the procedure utilized for the characterisation of each nucleotide sequence is described below. Selection of polynucleotides were chosen based on their upregulation in ovarian tumors and the selective nature of their expression in these tumors compared to other tissues as described above. The design of shRNA sequences was performed using web-based software that is freely available to those skilled in the art (Qiagen for example). These chosen sequences, usually 19-mers, were included in two complementary oligonucleotides that form the template for the shRNAs, i.e. the 19-nt sense sequence, a 9-nt linker region (loop), the 19-nt antisense sequence followed by a 5-6 poly-T tract for termination of the RNA polymerase III. Appropriate restriction sites were inserted at the ends of these oligonucleotides to facilitate proper positioning of the inserts so that the transcriptional start point is at a precise location downstream of the hU6 promoter. The plasmid utilized in all RNA interference studies, pSilencer 2.0 (SEQ. ID. NO. 17), was purchase from a commercial supplier (Ambion, Austin, Tex.). For each sequence selected, at least two different shRNA expression vectors were constructed to increase the chance of observing RNA interference.

Determination of knockdown effect is determined using TOV-21G or TOV-112D cells which are seeded in 6-well plates in OSE (Samouelian et al., 2004) containing 10% fetal bovine serum at a density of 600 000 cells/well, allowed to plate overnight and transfected with 1 μg of pSil-shRNA plasmid using the Fugene 6 reagent (Roche, Laval, QC). After 16 h of incubation, fresh medium is added containing 2 μg/ml puromycin (Sigma, St. Louis, Mo.) to select for stable transfectants. Control cells are transfected with a control pSil (sh-scr available at Ambion) that contains a scrambled shRNA sequence that displays homology to no known human gene. After approximately 4-5 days, pools and/or individual clones of cells are isolated and expanded for further analyses. The effectiveness of attenuation is verified in all shRNA cells lines. Total RNA is prepared by standard methods using Trizol™ reagent from cells grown in 6-well plates and expression of the target gene is determined by RT-PCR using gene-specific primers. First strand cDNA is generated using Thermoscript (Invitrogen, Burlington, ON) and semi-quantitative PCR is performed by standard methods (Qiagen, Mississauga, ON). 100% expression levels for a given gene is assigned to those found in the cell lines transfected with the control pSil plasmid (sh-scr).

The proliferative ability of each shRNA-expressing cell line is determined and compared to cells expressing the scrambled shRNA (control). Cell number is determined spectrophotometrically by MTT assay at 570 nm (Mosmann, 1983). After selection of stably shRNA expressing pools and expansion of the lines, 5 000 cells/well of each cell lines is plated in 48-well plates in triplicate and incubated for 4 days under standard growth conditions. Representative data from 2 experiments ±SEM is displayed and experiments are typically repeated at least three times to confirm the results observed.

The gene encoding the folate receptor 1, SEQ. ID. NO. 4 (0967A) (FIG. 3B, 0967A), which has been documented as being an important marker for ovarian cancer (Leamon and Low, 2001), is attenuated in TOV-21G cells, and marked growth inhibition is observed in the presence of the shRNAs (sh-1: SEQ. ID. NO. 34 and sh-2: SEQ. ID. NO. 35). This gives credibility to the approach used herein. Assays similar to the above are performed for SEQ ID NO:2, for fragments of SEQ ID NO:2 (e.g., those encoding the polypeptides described herein), for SEQ ID NO:2 variants and fragments.

### A Method for Determining the Requirement for Specific Genes in the Survival of Ovarian Cancer Cells

As a means of complementing the growth inhibition data that are generated with the stable TOV-21G cell lines, a colony survival assay is used to determine the requirement of the selected genes in the survival of the cancer cells. The ‘colony formation assay’ or ‘clonogenic assay’ is a classical test to evaluate cell growth after treatment. The assay is widespread in oncological research areas where it is used to test the proliferating power of cancer cell lines after radiation and/or treatment with anticancer agents. It is expected that the results obtained when analyzing the genes that were functionally important in ovarian cancer correlate between the growth inhibition study and the colony survival assay.

TOV-21G cells are seeded in 12-well plates at a density of 50 000 cells/well and transfected 24 h later with 1 μg of pSil-shRNA vector, the same plasmids used in the previous assay. The next day, fresh medium is applied containing 2 μg/ml puromycin and the selection of the cells is carried out for 3 days. The cells are washed and fresh medium without puromycin is added and growth continued for another 5 days. To visualize the remaining colonies, the cells are washed in PBS and fixed and stained simultaneously in 1% crystal violet/10% ethanol in PBS for 15 minutes at room temperature. Following extensive washing in PBS, the dried plates are scanned for photographic analysis.

### Other Oncology Indications

One skilled in the art will recognize that the sequences described in this invention have utilities in not only ovarian cancer, but these applications can also be expanded to other oncology indications where the genes are expressed. To address this, a PCR-based method is adapted to determine the expression pattern of all sequences described above in cancer cell lines isolated from nine types of cancer. The cancer types represented by the cell lines are leukemia, central nervous sytem, breast, colon, lung, melanoma, ovarian, prostate, and renal cancer (see Table 5). These RNA samples are obtained from the Developmental Therapeutics Program at the NCl/NIH. Using the same RAMP RNA samples that amplified from the total RNA samples obtained from the NCl, 500 μg of RNA is converted to single-stranded cDNA with Thermoscript RT (Invitrogen, Burlington, ON) as described by the manufacturer. The cDNA reaction is diluted so that 1/200 of the reaction is used for each PCR experiment. After trial PCR reactions with gene-specific primers designed against each SEQ. ID NOs. to be tested, the linear range of the reaction is determined and applied to all samples, PCR is conducted in 96-well plates using Hot-Start Taq Polymerase from Qiagen (Mississauga, ON) in a DNA Engine Tetrad from MJ Research. Half of the reaction mixture is loaded on a 1.2% agarose/ethidium bromide gel and the amplicons visualized with UV light. To verify that equal quantities of RNA is used in each reaction, the level of RNA is monitored with GAPDH expression.

TABLE 5
List of cancer cell lines from the NCI-60 panel

| Cell line | Cancer type |
|---|---|
| K-562 | leukemia |
| MOLT-4 | leukemia |
| CCRF-CEM | leukemia |
| RPMI-8226 | leukemia |
| HL-60(TB) | leukemia |
| SR | leukemia |
| SF-268 | CNS |
| SF-295 | CNS |
| SF-539 | CNS |
| SNB-19 | CNS |
| SNB-75 | CNS |
| U251 | CNS |
| BT-549 | breast |
| HS 578T | breast |
| MCF7 | breast |
| NCI/ADR-RES | breast |
| MDA-MB-231 | breast |
| MDA-MB-435 | breast |
| T-47D | breast |
| COLO 205 | colon |
| HCC-2998 | colon |
| HCT-116 | colon |
| HCT-15 | colon |
| HT29 | colon |
| KM12 | colon |
| SW-620 | colon |
| A549/ATCC | non-small cell lung |
| EKVX | non-small cell lung |
| HOP-62 | non-small cell lung |
| HOP-92 | non-small cell lung |
| NCI-H322M | non-small cell lung |
| NCI-H226 | non-small cell lung |
| NCI-H23 | non-small cell lung |
| NCI-H460 | non-small cell lung |
| NCI-H522 | non-small cell lung |
| LOX IMVI | melanoma |
| M14 | melanoma |
| MALME-3M | melanoma |
| SK-MEL-2 | melanoma |
| SK-MEL-28 | melanoma |
| SK-MEL-5 | melanoma |
| UACC-257 | melanoma |
| UACC-62 | melanoma |
| IGROV-1 | ovarian |
| OVCAR-3 | ovarian |
| OVCAR-4 | ovarian |
| OVCAR-5 | ovarian |
| OVCAR-8 | ovarian |
| SK-OV-3 | ovarian |
| DU-145 | prostate |
| PC-3 | prostate |
| 786-O | renal |
| A498 | renal |
| ACHN | renal |
| CAKI-1 | renal |
| RXF-393 | renal |
| SN-12C | renal |
| TK-10 | renal |
| UO-31 | renal |

One of skill in the art will readily recognize that orthologues for all mammals maybe identified and verified using well-established techniques in the art, and that this disclosure is in no way limited to one mammal. The term “mammal(s)” for purposes of this disclosure refers to any animal classified as a mammal, including humans, domestic and farm animals, and zoo, sports, or pet animals, such as dogs, cats, cattle, horses, sheep, pigs, goats, rabbits, etc. Preferably, the mammal is human.

The sequences in the experiments discussed above are representative of the NSEQ being claimed and in no way limit the scope of the invention. The disclosure of the roles of the NSEQs in proliferation of ovarian cancer cells satisfies a need in the art to better understand ovarian cancer disease, providing new compositions that are useful for the diagnosis, prognosis, treatment, prevention and evaluation of therapies for ovarian cancer and other cancers where said genes are expressed as well.

The art of genetic manipulation, molecular biology and pharmaceutical target development have advanced considerably in the last two decades. It will be readily apparent to those skilled in the art that newly identified functions for genetic sequences and corresponding protein sequences allows those sequences, variants and derivatives to be used directly or indirectly in real world applications for the development of research tools, diagnostic tools, therapies and treatments for disorders or disease states in which the genetic sequences have been implicated.

Although the present invention has been described herein above by way of preferred embodiments thereof, it maybe modified, without departing from the spirit and nature of the subject invention as defined in the appended claims.

TABLE 6
Differentially expressed sequences found in malignant ovarian cancer.

| | NCBI | | ORF | |
|---|---|---|---|---|
| | Unigene | | Nucleotide | |
| | #/Gene | | Positions/ | |
| Nucleotide | Symbol/Gene | Accession | Polypeptide | |
| Sequence No. | ID | Number | sequence No. | Function/Comments |
| SEQ ID NO. 1 | STAR clone | AK092936 | | Novel genomic hit |
| | | | | (see |
| | | | | PCT/CA2007/001134) |
| SEQ ID NO.: 2 | | | encoding SEQ ID | |
| | | | NOs.: 3, 22, 23 | |
| | | | and 24. | |
| SEQ ID NO: 3 | | | | +3 frame, position 1425-1859 |
| | | | | of SEQIDNO: 2, |
| | | | | (ERV3 variant) |
| SEQ ID NO. 4 | Hs.73769/ | NM_000802 | 26-799 | folate receptor 1 (adult); |
| | FOLR1/ | | encoding SEQ ID | mediate delivery of 5- |
| | 2348 | | NO.: 5 | methyltetrahydrofolate to |
| | | | | the interior of cells |

TABLE 7
List of additional sequences identification of plasmids,
oligonucleotides and shRNA oligonucleotides

| Sequence | | |
|---|---|---|
| Identification | name | Description |
| SEQ. ID. NO. 6 | OGS 364 | Oligo dT₁₁ + Not 1 + biotin |
| SEQ. ID. NO. 7 | OGS 594 | Oligonucleotide promoter tag 1 |
| SEQ. ID. NO. 8 | OGS 595 | Oligonucleotide promoter tag 1 |
| SEQ. ID. NO. 9 | OGS 458 | Oligonucleotide promoter tag 2 |
| SEQ. ID. NO. 10 | OGS 459 | Oligonucleotide promoter tag 2 |
| SEQ. ID. NO. 11 | OGS 494 | Primer for second-strand synthesis |
| | | from tag 1 |
| SEQ. ID. NO. 12 | OGS 302 | Primer for second-strand synthesis |
| | | from tag 2 |
| SEQ. ID. NO. 13 | OGS 621 | Oligonucleotide promoter |
| SEQ. ID. NO. 14 | OGS 622 | Oligonucleotide promoter |
| SEQ. ID. NO. 15 | pCATRMAN | Vector for STAR |
| SEQ. ID. NO. 16 | p20 | Vector for STAR |
| SEQ. ID. NO: 17 | pSilencer2.0 | Vector for shRNA |
| | vector | |
| SEQ. ID NO. 18 | OGS 1035 | Forward primer for SEQ ID NO. 4 |
| SEQ. ID NO. 19 | OGS 1036 | Reverse primer for SEQ ID NO. 4 |
| SEQ. ID. NO: 20 | OGS 1212 | Forward primer for SEQ ID NO. 1 |
| SEQ. ID. NO: 21 | OGS 1213 | Reverse primer for SEQ ID NO. 1 |
| SEQ. ID. NO: 22 | | Polypeptide encoded at +2 frame, |
| | | position 518-820 of SEQIDNO: 2 |
| | | (ERV3 variant) |
| SEQ. ID. NO: 23 | | Polypeptide encoded at +1 frame, |
| | | position 112-339 of SEQIDNO: 2 |
| | | (ERV3 variant) |
| SEQ. ID. NO: 24 | | Polypeptide encoded at +3 frame |
| | | position 3-179 of SEQIDNO: 2 |
| | | (ERV3 variant) |
| SEQ. ID. NO: 25 | | Polypeptide encoded by open reading |
| | | frame identified at position 1021 to |
| | | 1218 of SEQ ID NO: 2 |
| SEQ. ID. NO: 26 | | Polypeptide encoded by open reading |
| | | frame identified at position 1336 to |
| | | 1461 of SEQ ID NO: 2 |
| SEQ. ID. NO: 27 | | Polypeptide encoded by open reading |
| | | frame identified at position 120 |
| | | to 410 of the anti-sense strand |
| | | SEQ ID NO: 2 |
| SEQ. ID. NO: 28 | | Polypeptide encoded by open reading |
| | | frame identified at position 427 |
| | | to 639 of the anti-sense strand |
| | | SEQ ID NO: 2 |
| SEQ. ID. NO: 29 | | Polypeptide encoded by open reading |
| | | frame identified at position 1228 to |
| | | 1401 of the anti-sense strand SEQ ID |
| | | NO: 2 |
| SEQ. ID. NO: 30 | | Polypeptide encoded by open reading |
| | | frame identified at position 828 |
| | | to 980 of the anti-sense strand |
| | | SEQ ID NO: 2 |
| SEQ. ID. NO: 31 | | Polypeptide encoded by open reading |
| | | frame identified at position 1196 to |
| | | 1318 of the anti-sense strand SEQ ID |
| | | NO: 2 |
| SEQ. ID. NO: 32 | | Acc. No. AAP06678.1 |
| | | Acc. No. AAP29640.1 |
| SEQ. ID. NO: 33 | | |
| SEQ. ID. NO: 34 | sh-1 0967 | shRNA sequence for SEQ. ID. NO. 4 |
| SEQ. ID. NO: 35 | sh-2 0967 | shRNA sequence for SEQ ID NO. 4 |
| SEQ. ID. NO: 36 | OGS 315 | Forward primer for human GAPDH |
| SEQ. ID. NO: 37 | OGS 316 | Reverse primer for human GAPDH |
| SEQ. ID. NO: 38 | 0532.3-top | shRNA sequence for SEQ. ID. NO. 3 |
| | shRNA | |
| SEQ. ID. NO: 39 | 0532.4-top | shRNA sequence for SEQ. ID. NO. 3 |
| | shRNA | |

### REFERENCES

Jemal A, Murray T, Ward E, Samuels A, Tiwari R C, Ghafoor A, Feuer E J and Thun M J. Cancer statistics, 2005. CA Cancer J Clin 2005; 55:10-30.
Menon U, Skates S J, Lewis S, Rosenthal A N, Rufford B, Sibley K, Macdonald N, Dawnay A, Jeyarajah A, Bast R C Jr, Oram D and Jacobs I J. Prospective study using the risk of ovarian cancer algorithm to screen for ovarian cancer. J Clin Oncol. 2005 Nov. 1; 23(31):7919-26.
Bonome T, Lee J Y, Park D C, Radonovich M, Pise-Masison C, Brady J, Gardner G J, Hao K, Wong W H, Barrett J C, Lu K H, Sood A K, Gershenson D M, Mok S C and Birrer M J. Expression profiling of serous low malignancy potential, low grade, and high grade tumors of the ovary. Cancer Res 2005; 65:10602-10612.
Chambers, A. and Vanderhyden, B. Ovarian Cancer Biomarkers in Urine. Clin Cancer Res 2006; 12(2):323-327.
Berek et al. Cancer Medicine. 5th ed. London: B. C. Decker, Inc.; 2000. p. 1687-1720.
Bristow R. E. Surgical standards in the management of ovarian cancer. Curr Opin Oncol 2000; 12:474-480.
Brown E, Stewart M, Rye T, Al-Nafussi A, Williams A R, Bradburn M, Smyth J and Gabra H. Carcinosarcoma of the ovary: 19 years of prospective data from a single center. Cancer 2004; 100:2148-2153.
Shih, L-M and Kurman, R J. Molecular Pathogenesis of Ovarian Borderline Tumors: New Insights and Old Challenges. Clin Cancer Res 2005; 11(20):7273-7279.
Seidman J D, Russell P, Kurman R J. Surface epithelial tumors of the ovary. In: Kurman R J, editor. Blaustein's pathology of the female genital tract. 5th ed. New York: Springer-Verlag; 2002. pp. 791-904.
Mor G, Visintin I, Lai Y, Zhao H, Schwartz P, Rutherford T, Yue L, Bray-Ward P and Ward D C Serum protein markers for early detection of ovarian cancer. PNAS 2005; 102:7677-7682.
Kozak K R, Amneus M W, Pusey S M, Su F, Luong M N, Luong S A, Reddy S T and Farias-Eisner R. Identification of biomarkers for ovarian cancer using strong anion-exchange ProteinChips: potential use in diagnosis and prognosis. PNAS 2003; 100:12343-12348.
McIntosh M W, Drescher C, Karlan B, Scholler N, Urban N, Hellstrom K E and Hellstrom I. Gynecol Oncol. 2004 October; 95(1):9-15.
Woolas R P, Xu F J, Jacobs I J, Yu Y H, Daly L, Berchuck A, Soper J T, Clarke-Pearson D L, Oram D H and Bast R C Jr. Elevation of multiple serum markers in patients with stage I ovarian cancer. J Natl Cancer Inst. 1993 Nov. 3; 85(21):1748-51
Schorge J O, Drake R D, Lee H, Skates S J, Rajanbabu R, Miller D S, Kim J H, Cramer D W, Berkowitz R S and Mok S C. Osteopontin as an adjunct to CA125 in detecting recurrent ovarian cancer. Clin. Cancer Res. 10; 3474-3478.
Gorelik E, Landsittel D P, Marrangoni A M, Modugno F, Velikokhatnaya L, Winans M T, Bigbee W L, Herberman R B and Lokshin A E. Multiplexed Immunobead-Based Cytokine Profiling for Early Detection of Ovarian Cancer Cancer Epidemiol Biomarkers Prey. 2005 April; 14(4):981-7. Mack et al., 2003. U.S. Patent Application 20030124579.
Monahan et al., 2003. U.S. Patent Application 20030087250.
Wonsey et al., 2006. U.S. Patent Application 20060014686.
Santin A. D., 2006. U.S. Patent Application 20060078941.
Santin A. D., 2006. U.S. Patent Application 20060084594.
Jazaeri et al., 2005. U.S. Patent Application 20050095592.
Monahan et al., 2005. U.S. Patent Application 20050214831.
Gordon et al., 2003. U.S. Patent Application 20030219760.
Mor et al., 2005. U.S. Patent Application 20050214826.
Jhaveri et al., 2006. Antisense oligonucleotides targeting folate receptor alpha, and use thereof. U.S. Pat. No. 7,030,236.
Agrawal N, Dasaradhi P V, Mohmmed A, Malhotra P, Bhatnagar R K and Mukherjee S K. RNA interference: biology, mechanism, and applications. Microbiol Mol Biol Rev. 2003 December; 67(4):657-85.
Brummelkamp T R, Bernards R and Agami R. A system for stable expression of short interfering RNAs in mammalian cells. Science 2002. 296: 550-553.
Elbashir S M, Harborth J, Lendeckel W, Yalcin A, Weber K, and Tuschl T. Duplexes of 21-nucleotide RNAs mediate RNA interference in cultured mammalian cells. Nature 2001. 411: 494-498.
Hannon G J. RNA interference. Nature 2002. 418: 244-251.
Leamon C P and Low P S. Folate-mediated targeting: from diagnostics to drug and gene delivery. Drug Discov Today 2001. 6: 44-51.
Mosmann T. Rapid colorimetric assay for cellular growth and survival: application to proliferation and cytotoxicity assays. J Immunol Methods. 1983. 65: 55-63.
Provencher D M, Lounis H, Champoux L, Tetrault M, Manderson E N, Wang J C, Eydoux P, Savoie R, Tonin P N and Mes-Masson A M. Characterization of four novel epithelial ovarian cancer cell lines. In Vitro Cell Dev Biol Anim 2000. 36: 357-361.
Samouelian V, Maugard C M, Jolicoeur M, Bertrand R, Arcand S L Tonin P N, Provencher D M, and Mes-Masson A M. Chemosensitivity and radiosensitivity profiles of four new human epithelial ovarian cancer cell lines exhibiting genetic alterations in BRCA2, TGFbeta-RII, KRAS2, TP53 and/or CDNK2A. Cancer Chemother Pharmacol 2004. 54: 497-504.

1-25. (canceled)

26. A method for treating cancer comprising administering a polypeptide having at least 75% identity with SEQ ID NO.:3 or a fragment thereof, a nucleic acid capable of inhibiting the expression of the polypeptide or an antibody or antigen binding fragment specifically binding to the polypeptide to a mammal in need.

27. (canceled)

28. An isolated antibody or antigen binding fragment thereof comprising a light chain variable region and a heavy chain variable region capable of specific and non-covalent binding to a polypeptide having at least 75% identity to SEQ ID NO.:3 wherein said antibody or antigen binding fragment specifically binds to cancer cells.

29. (canceled)

30. The antibody or antigen binding fragment of claim 28, wherein the cancer cells comprise ovarian cancer cells.

31-36. (canceled)

37. A pharmaceutical composition comprising the antibody or antigen binding fragment of claim 28 and a carrier.

38-41. (canceled)

42. A method of detection or diagnosis of cancer, the method comprising administering the pharmaceutical composition of claim 37 to a mammal in need.

43-67. (canceled)

68. The method of claim 26, wherein the antibody or antigen binding fragment specifically binds to cancer cells.

69. The method of claim 68, wherein the cancer cells comprises ovarian cancer cells.

70. The method of claim 68, wherein the antibody or antigen binding fragment comprises a therapeutic moiety.

71. The method of claim 26, wherein the antibody or antigen binding fragment inhibits the activity of SEQ ID NO.:3 in ovarian cancer cells.

72. The method of claim 68, wherein the mammal suffers from ovarian cancer.

73. The antibody or antigen binding fragment of claim 28, wherein the antibody or antigen binding fragment specifically binds to ovarian cancer cells.

74. The antibody or antigen binding fragment of claim 28, further comprising a therapeutic moiety or a detectable moiety.

75. The antibody or antigen binding fragment of claim 28, wherein the antibody or antigen binding fragment inhibits the activity of SEQ ID NO.:3 in ovarian cancer cells.

76. The pharmaceutical composition of claim 37, wherein the cancer cells comprises ovarian cancer cells.

77. The pharmaceutical composition of claim 37, wherein the antibody or antigen binding fragment specifically binds to ovarian cancer cells.

78. The pharmaceutical composition of claim 37, wherein the antibody or antigen binding fragment further comprises a therapeutic moiety or a detectable moiety.

79. The pharmaceutical composition of claim 37, wherein the antibody or antigen binding fragment inhibits the activity of SEQ ID NO.:3 in ovarian cancer cells.

80. The method of claim 42, wherein the cancer cells comprises ovarian cancer cells.

81. The method of claim 42, wherein the antibody or antigen binding fragment specifically binds to ovarian cancer cells.

82. The method of claim 42, wherein the antibody or antigen binding fragment further comprises a detectable moiety.
